SEQUENCE LISTING (16 sequences)

SEQ ID NO: 1 - DNA, 18 bp, Homo sapiens
ttgacccgtctccatcgg

SEQ ID NO: 2 - DNA, 18 bp, Homo sapiens
tgcctcctgacccctcct

SEQ ID NO: 3 - DNA, 25 bp, Homo sapiens
ccccagcccctatctccaagagaca

SEQ ID NO: 4 - DNA, 18 bp, Homo sapiens
tgagcgcggctacagctt

SEQ ID NO: 5 - DNA, 22 bp, Homo sapiens
tccttaatgtcacgcacgattt

SEQ ID NO: 6 - DNA, 18 bp, Homo sapiens
accaccacggccgagcgg

SEQ ID NO: 7 - DNA, 28 bp, Homo sapiens
aagaaggaaagatttgtaccagttgaga

SEQ ID NO: 8 - DNA, 26 bp, Homo sapiens
ggaaacccatcaatcaagaatttatt

SEQ ID NO: 9 - DNA, 28 bp, Homo sapiens
agagggaaatggatcagacaatggctgc

SEQ ID NO: 10 - DNA, 38 bp, Homo sapiens
tttattggtaccacgcgtacaaaggcaagtaggtgagg

SEQ ID NO: 11 - DNA, 38 bp, Homo sapiens
gcgattggtaccacgcgtctttccataatcaaaagtcc

SEQ ID NO: 12 - DNA, 37 bp, Homo sapiens
aataaaaagcttctcgagacaccgcgcctcggccgga

SEQ ID NO: 13 - DNA, 23 bp, Homo sapiens
aattatttaagggttggaggaaa

SEQ ID NO: 14 - DNA, 24 bp, Homo sapiens
ctacaacaaaaaactaaaacccaa

SEQ ID NO: 15 - DNA, 22 bp, Homo sapiens
tatttaagggttggaggaaagt

SEQ ID NO: 16 - DNA, 245 bp, Homo sapiens
tggcgcggaccgcacgagtgtcgccgagacgccgccgccgtgcgaagctcctccccttccgacagggccgcggacgcccgggcagccacggcggcggggccgcggcgggcgccggctcagcccgcccctttctcccgccgcctccccgccccgccccgcgccgcgccggccgctgtcagctccctcagcgtccggccgaggcgcggtgtatgctgagccgctgccgcagcgggctgctccacgtc

(19) United States
(12) Patent Application Publication
(10) Pub. No.: US 2010/0151004 A1
(43) Pub. Date: Jun. 17, 2010

(54) MODULATION OF DRUG SENSITIVITY

(75) Inventors: Debabrata Banerjee, Bellerose, NY (US); Joseph R. Bertino, Branford, CT (US); Rita Humeniuk, Gaithersburg, MD (US); Prasun J. Mishra, Gaithersburg, MD (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: UNIVERSITY OF MEDICINE AND DENTISTRY OF NEW JERSEY, Somerset, NJ (US)

(21) Appl. No.: 12/529,916

(22) PCT Filed: Mar. 7, 2008
(86) PCT No.: PCT/US2008/003027

§ 371 Date: Feb. 8, 2010

Related U.S. Application Data
(60) Provisional application No. 60/905,344, filed on Mar. 7, 2007.
(60) Provisional application No. 60/926,251, filed on Apr. 26, 2007.

(51) Int. Cl. A61K 9/127; A61K 31/706; A61K 31/352; A61K 31/195; A61K 31/513; A61K 31/7072; C12Q 1/68; G01N 33/53; A61P 35/00

(52) U.S. Cl. 424/450; 514/43; 514/456; 514/563; 514/49; 514/274; 514/50; 435/6; 435/7.1

(57) ABSTRACT

Methods for the treatment of disorders, including cancer, are described which include administering to a subject a DNA methylation inhibitor and an antineoplastic agent. Compositions containing a DNA methylation inhibitor and an antineoplastic agent, which are useful in treating disorders including cancer, are also described. Additionally, methods useful for developing a prognosis for or diagnosing a subject's development of resistance to treatment with a chemotherapeutic agent are described.

### CROSS-REFERENCE TO RELATED APPLICATIONS

This application claims benefit of U.S. Provisional Application No. 60/905,344, filed Mar. 7, 2007, and of U.S. Provisional Application No. 60/926,251, filed Apr. 26, 2007. The disclosures of each of these applications are hereby incorporated by reference herein, in their entireties.

### FIELD OF THE INVENTION

This invention relates generally to the diagnosis and treatment of conditions including cancer, and particularly to compositions and methods useful for improved efficacy of treatment with chemotherapeutic agents.

### BACKGROUND OF THE INVENTION

5-Fluorouracil (5-FU) has been used to treat colorectal, breast and gastric cancer for over 50 years. Initially it was used as a single agent and more recently in combination with oxaliplatin, irinotecan or bevacizumab (Avastin) that enhances its activity.

Colorectal cancer is the second leading cause of cancer-related deaths in the United States. 5-Fluorouracil (5-FU) has been the drug of choice in the clinic for the past several decades to treat patients diagnosed with this disease. In recent years, oxaliplatin and irinotecan have been used in combination with 5-FU and response rate and overall survival of patients with advanced disease has doubled. The response rate to 5-FU is typically less than 30% and intrinsic or acquired resistance to the drug is the main obstacle to successful treatment, especially in case of metastatic cancer where drug resistance is thought to cause treatment failure in over 90% of patients. Analysis of colorectal cancer hepatic metastasis of patients previously exposed to 5-FU showed a high frequency of low or undetectable expression of UMPK.

Two delivery methods of 5-FU are relevant from the clinical point of view: bolus short term infusions of high doses of the drug given weekly or continuous infusions of low doses days to weeks). The metabolism and mechanism of 5-FU action differs depending upon dose and schedule of administration. Continuous infusion results in 5-FU anabolism mainly to FdUMP, which then acts as a tight binding inhibitor of thymidylate synthase (TS) and inhibits DNA synthesis. Evidence has accumulated to support the notion that bolus treatment results in drug anabolism mainly to FUTP and is incorporated into RNA and interferes with normal pre-rRNA processing. It may be expected that resistance related to each mode of 5FU administration would differ and this is consistent with the observation that HCT-8 colon carcinoma cells resistant to bolus 5-FU treatment remain sensitive to continuous 5-FU exposure. The mechanism of resistance to 5-FU prolonged treatment is well characterized and it seems to be mainly related to an increased level of thymidylate synthase (TS), while resistance to bolus 5-FU remains to be elucidated. Previous studies indicated that cells resistant to bolus 5FU (HCT-8/4hFU) showed significantly decreased incorporation of the drug into RNA.

UMPK (also known as UMP/CMP kinase) catalyzes the transfer of the phosphate group to UMP, CMP and dCMP using ATP as a cofactor in the presence of magnesium. This enzyme is crucial for de-novo and salvage synthesis of pyrimidine nucleotides and no other enzyme with the same substrate specificity as UMP/CMP kinase, has been identified thus far. Besides its biological function, UMPK plays a very important role in the activation of nucleoside analogs used as anticancer or antiviral drugs, including 5-FU, zebularine, 1-β-D-arabinofuranosylcytosine (AraC), 2′,2′-difluorodeoxycytidine (gemcitibine), β-D-2′,3′-dideoxycytidine (ddC), β-L-2′,3′-dideoxy-3′-thiocytidine (3-TC), and 2′3′-deoxy-3′-azidothymidine (AZT) (Pasti C, Gallois-Montbrun S, Munier-Lehmann H, Veron M, Gilles A M, Deville-Bonne D. Reaction of human UMP-CMP kinase with natural and analog substrates. Eur J Biochem 2003 April; 270(8):1784-90). Importantly, UMPK is thought to be a rate-limiting enzyme in these processes, based on the observation of accumulation of UMP, CMP and its analogs in the cell. Despite the important function carried out by UMPK, little is known about the regulation of this enzyme. The cDNA for human UMPK has been cloned based on its homology with pig UMPK (Van Rompay A R, Johansson M, Karlsson A. Phosphorylation of deoxycytidine analog monophosphates by UMP-CMP kinase: molecular characterization of the human enzyme. Mol Pharmacol 1999 September; 56(3):562-9; Liou J Y, Dutschman G E, Lam W, Jiang Z, Cheng Y C. Characterization of human UMP/CMP kinase and its phosphorylation of D- and L-form deoxycytidine analogue monophosphates. Cancer Res 2002 Mar. 15; 62(6):1624-31).

A limited number of 5-FU response predictive biomarkers have been identified in colorectal cancer and their clinical utility still remains controversial. A high level of thymidylate synthase and its promoter polymorphism provides a mechanism of resistance to fluoropyrimidines. A low expression of thymidine phosphorylase gene and high expression of dihydropyrimidine dehydrogenase correlate with resistance to 5-FU. p53 status has been studied as a predictor of responsiveness to cancer chemotherapy but conflicting results have been reported in case of response to 5-FU.

Epigenetic events contribute significantly to the development and progression of cancer through inactivation of tumor suppressor, DNA repair genes and growth regulatory microRNAs. These alterations have been proposed to precede or even facilitate genetic mutations or genomic instability and chromosome translocations that have long been associated with carcinogenesis. It is of particular importance to identify genes or chromosomal regions, the epigenetic alterations of which foster clonal expansion and accumulation of additional genetic modifications. Epigenetic alterations are known to involve mechanisms including methylation of DNA and/or resetting the complex code of histones that in turn leads to changes in chromatin structure that affects transcription. The molecular determinants that result in chromatin change in tumor cells are only beginning to be elucidated.

The best understood is the role of promoter DNA methylation in silencing of tumor suppressor genes. DNA methylation occurs through covalent addition of a methyl group to cytosine in the cytosine-guanosine dinucleotide context (CpG). Long stretches of DNA that have a high occurrence frequency of CpG sites are called CpG islands and unlike in the cancer cell they remain mostly unmethylated in the normal cell. CpG islands are mostly found in or near promoters of genes where the transcription is initiated. Methylation inhibits the process of transcription by directly interfering with a binding of certain transcription factors (activators). It also works in close collaboration with chromatin remodeling complexes from the Polycomb and Trithorax families that influence chromatin structure in the vicinity of the gene promoter.

Promoter CpG island methylator phenotype (CIMP) has been described for a distinct subset of colorectal cancers and adenomas, indicating that DNA methylation in cancer does not happen in a stochastic manner but involves a programmed series of events. Moreover, incremental methylation in normal colon has been shown to be age-dependent; and a stepwise increase in the average number of genes methylated per sample was identified during colorectal carcinogenesis. A substantial number of genes with promoter hypermethylation have been identified in colorectal cancer. Loss of function of the Adenomatous Polyposis Coli (APC), a tumor suppressor gene linked to familial colorectal cancer as well as the Wnt antagonist (DKK-1), have been reported to be silenced due to methylation. Methylation of CpG sites in the mismatch repair gene hMLH1 promoter are frequently present in sporadic colorectal cancer with microsatellite instability (MSI). Methylation of the hMLH1 promoter interferes with its binding to transcription factor CBF and inhibits gene expression. The cell cycle regulator gene p16 was also reported to be hypermethylated in 24 out of 84 T3N0M0 stage primary colorectal cancers. The presence of p16 hypermethylation predicts for a shorter survival. Additionally, CpG island promoter hypermethylation and silencing of nucleotide-releasing factor (RASGRF2), apoptosis-associated TF (BHLHB9) and homeobox gene (HOXD1) were discovered using a search aiming to define the colorectal cancer hypermethylome.

Multiple changes in the methylation of the CpG islands have been observed following drug selection in ovarian cancer. Methylation of the proapoptotic factors Apaf-1 and Caspase-8 has been reported to be associated with resistance to doxorubicin, etoposide and cisplatin. MDR1 was found to be progressively demethylated during the course of chemotherapy of acute myeloid leukemias and was correlated with overexpression of P-gp. Analysis of methylation of DNA mismatch repair gene hMLH1 revealed therapy related acquisition of methylation of this gene in 25% of patients and this was correlated with poor overall survival for ovarian cancer patients. MLH1 promoter methylation has also been shown to be associated with loss of DNA mismatch repair in colon cancer and resistance to 5-FU. Epigenetic driven changes in gene expression may provide a rapid and heritable means by which tumor cells can adapt to stress in the environment that cytotoxic drug therapy induces.

5-Azadeoxycytidine (DAC) is a hypermethylation inhibitor approved for the treatment of myelodysplastic syndrome (MDS) (Kantarjian H, Issa J P, Rosenfeld C S, et al. Decitabine improves patient outcomes in myelodysplastic syndromes: results of a phase III randomized study. Cancer 2006 Apr. 15; 106(8):1794-803). When used at lower doses, it reactivates tumor suppressor genes and tumor antigens that are silent due to methylation without cytotoxicity (Schrump D S, Fischette M R, Nguyen D M, et al. Phase I study of decitabine-mediated gene expression in patients with cancers involving the lungs, esophagus, or pleura. Clin Cancer Res 2006 Oct. 1; 12(19):5777-85). Despite this observation, only a limited number of clinical trials exploring the potential of DAC alone or in combination with existing chemotherapy in the treatment of solid tumors have been reported. It has been shown that DAC-mediated reactivation of hMLH1 expression reverses drug resistance in human tumor xenografts. Subsequently, a clinical trial has been initiated exploring the combination of DAC and carboplatin in solid tumors.

The foregoing underscores the importance of elucidating the role of UMPK in the development of resistance to chemotherapeutic drugs such as fluoropyrimidines, as well as the importance of determining the role of epigenetic events in the development of such resistance. To date, there remains a need for improved treatment for certain conditions, including cancers, in which patients develop resistance to chemotherapeutic agents.

### BRIEF SUMMARY OF THE INVENTION

In certain aspects, the invention relates to methods for the treatment of cancer which comprises administering to a subject in need thereof a therapeutically effective amount of a) a DNA methylation inhibitor and b) an antineoplastic agent.

Additional aspects of the invention relate to methods for increasing the efficacy of a nucleoside analog comprising the step of administering to a subject in need thereof a therapeutically effective amount of a) a DNA methylation inhibitor and b) the nucleoside analog.

Further aspects of the invention relate to compositions for use in treating cancer comprising: a) a DNA methylation inhibitor and b) an antineoplastic agent.

Additional aspects of the present invention are directed to methods of making compositions according to embodiments of the invention.

In further aspects, the invention relates to methods for developing a prognosis for or diagnosing a subject's development of resistance to treatment with an antineoplastic agent, the method comprising the steps of:

a) obtaining a sample from the subject;

b) measuring in the sample the level of expression of UMPK; and

c) comparing the level of expression of UMPK of the sample with that of a standard.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates a model showing that 5-FU has two different modes of action depending upon dose and schedule of administration. 5-FU activation to fluorouridine triphosphate (FUTP) and its incorporation into RNA is favored in bolus treatment, and 5-FU activation to fluorodeoxyuridine monophosphate (FdUMP) that inhibits activity of thymidylate synthase (TS) utilizing methylene tetrahydrofolate as a cofactor is favored in continuous exposure. Liver is the primary place of 5-FU degradation and requires Dihydropyrimidine dehydrogenase (DPD) activity. TP: thymidine phosphorylase; UP: uridine phosphorylase; TK: thymidine kinase; UK: uridine kinase; OPRTase: orotate phosphoribosyltransferase; UMPK: UMP kinase; NDPK: nucleoside diphosphate kinase; RR: ribonucleotide reductase; dUTPase: dUTP pyrophosphatase.

FIG. 2 illustrates that bolus 5-FU resistant HCT-8 cells that have lower expression of UMPK remain sensitive to continuous exposure to 5-FU. HCT-8/P and HCT-8/4hFU cells were exposed to bolus (A) or continuous 5-FU treatment (B). UMPK mRNA (C) and protein (D) levels were assayed and expressed as a relative fold change (Q-RT-PCR) or a percentage (Western Blotting). Experiments were done in triplicates and nonlinear regression with dose-response curve fitting and t-test comparing IC₅₀ values or relative fold change was performed to determine statistical significance.

FIG. 3 illustrates that liver metastasis from patients previously exposed to bolus 5-FU showed higher incidence of decreased UMPK levels. (A): UMPK mRNA level was quantitated in 10 samples of patients not previously exposed to bolus 5-FU and 19 samples of 5-FU treated patients. Expression was normalized to β-actin as described in Materials and Methods. Mean value±Standard Deviation is shown. Student-T-test was used to assay statistical significance. (B): UMPK protein level in samples of 5-FU treated patients (lane 1-9) and 5-FU untreated patients (lane 10-13). (C): Normalized UMPK protein level. Samples were averaged and Mean value±Standard Deviation is shown.

FIG. 4 illustrates that up-regulation of UMPK in HCT-4hFU cells restores its sensitivity to bolus 5-FU but not effect sensitivity to continuous exposure. (A): Dose response curves of UMPK cDNA transfected clones and empty vector to bolus 5-FU exposure (B): Dose response curves of UMPK cDNA transfected clones and empty vector to continuous 5-FU exposure (C): UMPK mRNA level in selected UMPK cDNA transfected clones as compared to empty vector transfected clones and untransfected HCT-8/4hFU cells. Clones were averaged and Mean±Standard Deviation is shown. (D): UMPK protein level in selected UMPK cDNA transfected clones (lane 2-4) and vector alone clones (lane 5-7). Experiments were done in triplicates and nonlinear regression with dose-response curve fitting and t-test comparing IC₅₀ values or relative fold change was performed to determine statistical significance.

FIG. 5 illustrates that down-regulation of UMPK in HCT-8/P cells induces resistance to bolus 5-FU but not continuous exposure. (A): Efficiency of siRNA-mediated down-regulation of UMPK protein as compared to scrambled siRNA and oligofectamine alone. (B): Bolus 5-FU dose response curves of HCT-8/P cells transfected with UMPK designed siRNA (siRNA#3 at 40 nM) as compared to untransfected cells. (C): Continuous 5-FU dose response curves of HCT-8/P cells transfected with UMPK designed siRNA (siRNA#3 at 40 nM) as compared to untransfected cells. (D): Bolus 5-FU dose response curves of HCT-8/P cells transfected with scrambled siRNA or oligofectamine alone as compared to untransfected cells. Experiments were done in triplicates and nonlinear regression with dose-response curve fitting and t-test comparing IC₅₀ values was performed to determine statistical significance.

FIG. 6 illustrates that HCT-8/4hFU cells are cross-resistant to 5-FUR and down-regulation of UMPK in HCT-8/P cells induces resistance to 5-FUR. (A): Dose response cures of HCT-8/P and HCT-8/4hFU cells exposed to 5-FUR. (B): Dose response curves of HCT-8/P cells transfected with UMPK designed siRNA (siRNA#3 at 20 nM) or combination of siRNA #1 and #3 at 20 nM each) to 5FUR. (C): Dose response curves of HCT-8/P cells transfected with scrambled siRNA or oligofectamine alone to 5FUR.

FIG. 7 illustrates that pretreatment of HCT-8/4hFU cells with low-doses of DAC restores its sensitivity to bolus 5-FU and 5-FUR treatments. (A): HCT-8/P and HCT-8/4hFU cells were exposed to DAC for 24 h and cytotoxicity was assayed 4 days later. (B): HCT-8/4hFU cells were exposed to 0.02, 0.1 or 0.5 μM DAC for 24 h and than treated for 4 hours with various concentrations of 5-FU (125 μM is shown) or 5-FUR (10 μM is shown) (C) Much less of an effect of DAC on sensitivity of HCT-8/P cells to 5-FUR was observed (D).

FIG. 8 illustrates DAC enhancement of 5-FU activity in a mouse colorectal cancer xenograft model. Mice bearing HCT-8/4hFU tumor (A) & (C) or HCT-8/P tumor HCT-8/P (B) & (D) were treated with DAC alone (3×0.5 mg/kg/course) (empty triangle), or DAC followed by bolus 5-FU (1×50 mg/kg/course) (cross), bolus 5-FU alone (1×50 mg/kg/course) (open square), or saline (closed square) as a control. Animals received either one (A) & (B) (8-10 animals/group) or two courses of treatment (C) & (D) (5-8 animals/group). Average Tumor Volume for all of the animals±Standard Deviations are shown. Two independent experiments are presented. Repeated Measure ANOVA test was used to assess statistical significance (p values for each group is given next to the figure legend).

FIG. 9 illustrates that DAC treatment results in elevated UMPK levels. UMPK mRNA (A) and protein (B) levels were assayed in HCT-8/4hFU cells following 24 h exposure to DAC. Mice bearing HCT-8/4hFU tumors were given one, two or three courses of DAC alone treatment (3×0.5 mg/kg/course). 24 h following DAC injection tumors were harvested and UMPK mRNA level was compared to tumors harvested from untreated animals (C). Alternatively, the average induction of UMPK in the all of the animals from the experimental groups for which tumor volume is presented in FIGS. 8C & 8D was quantitated and expressed as fold change relative to the UMPK mRNA level in untreated HCT-8/4hFU tumors (D). Mean value±Standard Deviation are shown.

FIG. 10 illustrates the cloning and expression of UMPK promoter region. (A): Location of UMPK gene denoted here as CMPK on chromosome 1. (B): Strategy used to clone UMPK promoter into pGL-3 vectors. Two forward and one reverse primers were designed to amplify 1000 bp and 2000 bp upstream of the first translation start site in exon 1 of UMPK gene. (C): UMPK promoter activity as compared to SV40 promoter (pGL-3 control vector). HCT-8/P or HCT-8/4hFU cells were transfected with constructs containing 1000 bp regions amplified from HCT-8/P or HCT-8/4hFU genomic DNA and cloned into the pGL-3 enhancer vector.

FIG. 11 illustrates: (A): Prediction of a CpG island located within the cloned 1000 bp region containing UMPK promoter and the first exon. Observed/Expected CpG ratio (upper panel), percentage of CpG dinucleotide (middle panel) and predicted CpG island (lower panel) are shown. (B): Sequence of UMPK promoter region in which DNA methylation was analyzed (SEQ ID NO: 16). Methylation of a total 42 CpG sites was analyzed. CpG sites are shown in red with the representative numbers shown above each site. Highlighted in blue is the beginning of UMPK transcript. (C): Average methylation across all of the 42 CpG sites analyzed detected in HCT-8/P and HCT-8/4hFU, as well as HCT-8/4hFU cells treated with 0.5 μM DAC for 24 h. Median±minimum and maximum values observed are shown.

FIG. 12 illustrates that HCT-8/P cells pretreated with DAC do not show increased sensitivity to 5-FU (A) or increased UMPK mRNA levels (B).

FIG. 13 illustrates that the decrease in UMPK mRNA in HCT-8/4hFU cells does not appear to be attributed to decreased UMPK mRNA half-life. Actinomycin D (A) or DRB (B) was used to inhibit transcription.

### DETAILED DESCRIPTION OF THE INVENTION

In certain embodiments, the present invention is directed to methods for the treatment of certain conditions which comprise administering to a subject in need thereof a therapeutically effective amount of at least one DNA methylation inhibitor and at least one antineoplastic agent. In certain embodiments, the condition to be treated is cancer. According to certain aspects of the invention, the cancer may include, but is not limited to, colorectal cancer, gastric cancer, or breast cancer.

In certain embodiments of the invention, DNA methylation inhibitors may include, but are not limited to, 5-azacytidine, 5-azadeoxycytidine, zebularine, epigallocatechin-3-gallate, 4-aminobenzoic acid derivatives, or psammaplins. Preferably, the DNA methylation inhibitor may be 5-azadeoxycytidine.

In certain embodiments of the invention, the antineoplastic agent may be a fluoropyrimidine. Preferably, the antineoplastic agent may be 5-fluorouracil or 5-fluorouridine. In certain preferred embodiments, a fluoropyrimidine may be administered to the subject on a bolus dosing schedule.

In certain aspects, the invention is directed to methods for increasing the efficacy of a nucleoside analog comprising the step of administering to a subject in need thereof a therapeutically effective amount of at least one DNA methylation inhibitor and at least one nucleoside analog.

In certain embodiments of the invention, examples of nucleoside analogs include, but are not limited to, 5-fluorouracil, 5-fluorouridine, zebularine, 1-β-D-arabinofuranosylcytosine (AraC), 2′,2′-difluorodeoxycytidine (gemcitibine), β-D-2′,3′-dideoxycytidine (ddC), β-L-2′,3′-dideoxy-3′-thiocytidine (3-TC), and 2′3′-deoxy-3′-azidothymidine (AZT). In certain embodiments, a fluoropyrimidine, including, but not limited to, 5-fluorouracil or 5-fluorouridine, may be administered on a bolus dosing schedule.

In other embodiments, the invention is directed to methods for developing a prognosis for or diagnosing a subject's development of resistance to treatment with an antineoplastic agent, such a method comprising the steps of: a) obtaining a sample from the subject; b) measuring in the sample the level of expression of UMPK; and c) comparing the level of expression of UMPK of the sample with that of a standard. A lower level of expression of UMPK compared to that of the standard may indicate the development of resistance to treatment with antineoplastic agents.

The standard level of expression of UMPK may include, for example: the level of expression of UMPK in a sample obtained from a healthy part of the subject's body, a sample obtained from a healthy individual, a previous sample obtained from the subject, or known levels of UMPK expression in a healthy individual.

Quantitation of UMPK expression levels may be achieved by measuring levels of UMPK mRNA or UMPK protein expressed in the cells of the sample. UMPK expression levels may be detected prior to, during, or post administration of any of the above-described agents, i.e., the DNA methylation inhibitor, and the antineoplastic agent. UMPK expression levels may be detected prior to the administration of an agent so as to ascertain severity or stage of the disease. Further, the UMPK expression levels may be monitored throughout the course of the treatment to check the efficacy of the treatment and/or prognosis of the disease.

In certain embodiments, the level of expression of UMPK in the sample may, for example, be measured by assaying the amount of UMPK RNA in the sample. The level of expression of UMPK in the sample may also, for example, be measured by assaying the amount of UMPK protein in the sample.

Preferably, the antineoplastic agent may be a fluoropyrimidine. Preferably, the fluoropyrimidine may be 5-fluorouracil or 5-fluorouridine. In certain embodiments, the fluoropyrimidine may be administered on a bolus dosing schedule.

The levels of UMPK expression may further be used to determine the amount of DNA methylation inhibitors and/or anti-neoplastic agents to be administered. UMPK expression levels can be determined by taking a biopsy. The level of UMPK expression in the biopsy is then compared with a control level of expression. A control level of expression can be a level of expression in a tissue sample derived from another part of the patient's body, a tissue sample derived from a healthy individual, a previous sample taken from the patient, or known levels of UMPK expression in a healthy individual. Expression levels can be determined using any known technique including, but not limited to, Northern blots and Western blots. If the level of UMPK expression in the biopsy is greater, by a statistically significant amount, from the level of UMPK expression in a control, then the patient is treated with a therapeutically effective amount of any of the following: DNA methylation inhibitors, antineoplastic agents, or a combination thereof. Combination treatment may require smaller dosages due to the synergetic effect of any of the above compositions. The level of UMPK expression in a patient can be determined prior to treatment, during treatment or post treatment. UMPK expression may be useful in diagnosing a particular type of disease or stage of the disease, as well as to verify efficacy of treatment.

Other aspects of the invention are directed to methods of diagnosing the development of resistance to treatment with a nucleoside analog in a subject comprising the steps of: a) obtaining a first sample from the subject; b) measuring in the sample the level of expression of UMPK; c) administering at least one nucleoside analog to the subject; d) obtaining a second sample from the subject; e) measuring in the second sample the level of expression of UMPK; and f) comparing the level of expression of UMPK of the first sample with that of the second sample. The level of expression of UMPK may be assayed, for example, by measuring products of transcription and/or translation.

Examples of nucleoside analogs include, but are not limited to, 5-fluorouracil, 5-fluorouridine, zebularine, 1-β-D-arabinofuranosylcytosine (AraC), 2′,2′-difluorodeoxycytidine (gemcitibine), β-D-2′,3′-dideoxycytidine (ddC), β-L-2′,3′-dideoxy-3′-thiocytidine (3-TC), and 2′3′-deoxy-3′-azidothymidine (AZT). In certain embodiments, a fluoropyrimidine, including, but not limited to, 5-fluorouracil or 5-fluorouridine, may be administered on a bolus dosing schedule.

Another aspect of the invention is directed to compositions useful in treating certain conditions, for example, cancer. In certain aspects, the composition may comprise: at least one DNA methylation inhibitor and at least one antineoplastic agent. DNA methylation inhibitors may include, but are not limited to, 5-azacytidine, 5-azadeoxycytidine, zebularine, epigallocatechin-3-gallate, 4-aminobenzoic acid derivatives, and psammaplins. Preferably, the DNA methylation inhibitor may be 5-azadeoxycytidine. Preferably, the antineoplastic agent may be a fluoropyrimidine. Preferably, the antineoplastic agent may be 5-fluorouracil or 5-fluorouridine.

In certain preferred embodiments, the composition may further comprise a liposome. Liposomes, or phospholipid vesicles, have long been recognized as drug delivery vehicles. A number of liposomal drug formulations have been approved for the treatment of conditions including infectious diseases and cancer. (Mauer N, Fenske D B, Cullis P R. Developments in liposomal drug delivery systems. Expert Opin Biol Ther 2001; 6:923-47). The application of liposomes as a delivery vehicle for drug combinations, particularly in the treatment of cancer, may have several advantages. Although the interaction of chemotherapeutic agents at different drug ratios can be systematically studied in vitro, due to differential pharmacokinetic characteristics of different drugs, these ratios cannot be easily translated in vivo. Without intending to be bound by any theory or theories of operation, it is believed that the co-encapsulation of two active agents into liposomes may “synchronize” the distribution of the drugs if the drugs can be stably entrapped inside the liposomes. This may allow for a more direct translation of in vitro results to in vivo. (Lee, R. J., Liposomal delivery as a mechanism to enhance synergism between anticancer drugs, Mol Cancer Ther. 2006; 5:1639-1640). According to certain aspects of the invention, the components of the composition may be incorporated into liposomes using any suitable means known to the art.

Another aspect of the invention relates to methods for making a composition useful for treating cancer comprising the step of combining at least one DNA methylation inhibitor and at least one antineoplastic agent. In certain preferred embodiments, the method further comprising the step of encapsulating the DNA methylation inhibitor and antineoplastic agent in liposomes.

According to any of the above embodiments, the DNA methylation inhibitor may be a cytidine analog or derivative thereof. Examples of cytidine analogs or derivatives include, for example, 5-azacytidine and 5-aza-2′-deoxycytidine (“decitabine”). Other DNA methylation inhibitors that may be used are zebularine, epigallocatechin-3-gallate, 4-Aminobenzoic acid derivatives and psammaplins. In a preferred embodiment, the DNA methylation inhibitor is decitabine.

As used herein, “therapeutically effective amount” means the dosage that provides the specific pharmacological response for which the active agent is administered in a significant number of subjects in need of the relevant treatment. It is emphasized that a therapeutically effective amount of the active agent that is administered to a particular subject in a particular instance will not always be effective in treating the conditions described herein, even though such dosage is deemed to be a therapeutically effective amount by those of skill in the art.

As used herein, the term “subject” is used to mean an animal, preferably a mammal, including a human. The terms “patient” and “subject” may be used interchangeably. Thus, certain embodiments of the invention are directed to appropriate dosage forms useful in the administration of active pharmaceutical ingredients to a subject.

The DNA methylation inhibitors and anti-neoplastic agents may be delivered via various routes of administration. For example, they may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or co-administered in slow release dosage forms. In certain preferred embodiments, the active agents are administered via liposomal delivery.

In certain embodiments, compounds and methods may be provided in which a liposomal drug delivery mixture is used to deliver two types of drugs; such as, for example, a DNA methylation inhibitor and an antineoplastic agent. In certain preferred embodiments, fluoropyrimidine compounds, including, but not limited to, 5-fluorouracil and 5-fluorouridine, have the potential to be used in a liposomal drug delivery mixture in combination with a DNA methylation inhibitor. In certain embodiments, 5-fluorouridine may offer superior solubility characteristics that may make it particularly suited for use in liposomal delivery systems.

According to particular embodiments of the invention, clinical resistance to 5-FU in certain cancers may be overcome by including DNA methylation inhibitors in the therapeutic regimen. The results achieved by the present inventors provide new insights into mechanisms of resistance to 5FU in colorectal cancer as well as mechanisms of UMPK gene regulation. Results of the studies suggest that clinical resistance to 5-FU in gastrointestinal cancers may be overcome by including DNA methylation inhibitors such as 5-AZA-dC in the therapeutic regimen. The benefits of using 5-FU+5-azadeoxycitidin in combination can extend beyond the treatment of bolus 5-FU resistant colorectal cancer. Other drugs with similar mechanism of action to 5-azadeoxycytidine may be used in combination with 5-FU.

Despite the effect of 5-FU on TS inhibition and DNA synthesis, it has been reported that 5-FU may have a significant effect on RNA metabolism. Previous studies indicated that colorectal cancer cells resistant to bolus 5-FU showed significantly decreased incorporation of the drug into RNA. Closer examination of the pulse 5-FU resistant cell line suggested that it might be due to a lower expression of UMP kinase (UMPK), an enzyme that plays an important role in the activation of 5-FU to 5F-UTP and its incorporation into RNA. Taking into consideration the fact that bolus 5-FU treatment acts predominantly on RNA metabolism and UMPK is an essential enzyme for RNA synthesis, these data suggested that cells exposed to 5-FU down-regulate UMPK as a mechanism that allows them to escape subsequent exposures to the drug.

The present inventors have found that tumor samples (hepatic metastasis) isolated from patients clinically resistant to bolus 5-FU treatment exhibited decreased expression of this enzyme as compared to tumor samples of patients not previously treated. The present inventors have also found that over-expression of UMPK cDNA in HCT-8/4hFUR cells confers sensitivity to bolus 5FU and UMPK down-regulation using siRNA knockdown in sensitive cells (HCT-8/P) induces resistance to bolus 5FU treatment. Further, HCT-8/4hFU cells are cross-resistant to the treatment with 5-fluorouridine (5FUR), consistent with the current understanding of 5-FUR as a RNA directed drug.

A number of published studies utilized whole genome profiling in search of additional 5-FU resistance-mediating genes. Analysis of gene expression profiles of various cell lines resistant to 5-FU showed three-fold decrease in UMPK mRNA. It has also been shown that UMPK down-regulation is one of the descriptors of intermediate-resistance phenotype, metastasis potential stage, but not primary resistance to 5-FU. Genomic instability is one of the characteristics of colorectal cancer and the UMPK gene, mapped on chromosome 1p32, is often involved in the deletion of short arm of chromosome 1. However, data suggest that UMPK gene down-regulation may occur prior to the deletion. UMPK may serve as a novel biomarker for predicting clinical resistance to pulse 5-FU treatment. Low UMPK expression may be a potential biomarker for a more advanced or malignant tumor phenotype that is less responsive to chemotherapy.

Acquired and intrinsic resistance still remains a primary limitation to the clinical use of 5-Fluorouracil (5-FU). Contemporary to the emergence of the field of cancer epigenetics several genes that are hypermethylated and silenced have been identified in colorectal cancer. The present inventors hypothesized that acquired resistance to 5-FU may have an epigenetic origin and might be reversed. The present inventors have found a novel therapeutic approach to circumvent clinical resistance to bolus 5-FU. Pretreatment of 5-FU bolus resistant colorectal cancer cells with low-dose 5-azadeoxycytidine (DAC), an inhibitor of DNA hypermethylation, restored sensitivity to 5-FU as well as 5-fluorouridine (5-FUR) treatments. Moreover, treatment of nude mice bearing 5-FU resistant tumors with DAC overcomes tumors resistance to bolus 5-FU. DAC mediated restoration of 5-FU sensitivity coincides with increases in UMPK levels. Cloning of the UMPK putative promoter region indicated the presence of a CpG island proximal to the putative transcription start site. Examination of the methylation status of this CpG island using a pyrosequencing method showed enrichment in DNA methylation in 5-FU resistant cells as compared to sensitive cells. Accordingly, clinical resistance to 5-FU due to decreased UMPK in certain cancers, including but not limited to, colorectal cancer, may be overcome by including methylation inhibitors such as, for example, DAC.

The increase in UMPK level following DAC treatment was dose as well as time dependent and was observed only in case of the bolus 5-FU resistant tumors but not in the parental tumors. Sequencing and expression of the UMPK promoter region revealed no functional changes between the two cell lines. Evaluation of 42 CpG sites within the island surrounding the putative transcription start site showed modest enrichment in DNA methylation across this region in HCT-8/4hFU cells that reverted following DAC treatment.

While not wishing to be bound by any theory or theories of operation, it is believed that the methylation of UMPK plays a role in acquired resistance to 5-FU, and that resistance to bolus 5-FU in certain cancers may be overcome by combination therapy with methylation inhibitors.

### EXAMPLES

The following examples serve to more fully describe the manner of using the above-described invention. It is understood that these examples in no way serve to limit the true scope of this invention, but rather are presented for illustrative purposes.

### Materials and Methods

### In Vitro CRC Model

Human intestinal adenocarcinoma cells (HCT-8) were maintained in RPMI 1640 supplemented with 10% horse serum and 1 mM sodium pyruvate and antibiotics. The cells were cultured under standard cell culture conditions, i.e. humidified atmosphere of 95% air and 5% CO₂ at 37° C. Bolus 5-FU resistant colorectal cancer cell lines were developed in a way that most closely mimics the clinical situation under which this resistance occurs, as described (Aschele C, Sobrero A, Faderan M A, Bertino J R. Novel mechanism(s) of resistance to 5-fluorouracil in human colon cancer (HCT-8) sublines following exposure to two different clinically relevant dose schedules. Cancer Res 1992 Apr. 1; 52(7):1855-64; Pizzorno G, Handschumacher R E. Effect of clinically modeled regimens on the growth response and development of resistance in human colon carcinoma cell lines. Biochem Pharmacol 1995 Feb. 14; 49(4):559-65.) Cells were exposed to 10⁻⁴M of 5-FU for 4 hours at 14-21 day intervals. Between drug exposures, cells were allowed to recover in drug free culture medium. Drug resistant colonies were expanded as cell lines, and are designated HCT-8/4hFU.

### UMPK Up-Regulation

Human UMPK cDNA cloned in a mammalian expression vector pCR3.1 (Invitrogen, Carlsbad, Calif., USA) was kindly provided to us by Dr Y. C. Cheng (Liou J Y, Dutschman G E, Lam W, Jiang Z, Cheng Y C. Characterization of human UMP/CMP kinase and its phosphorylation of D- and L-form deoxycytidine analogue monophosphates. Cancer Res 2002 Mar. 15; 62(6):1624-31). HCT-8/4hFUR cells were transfected with 2 mg of plasmid DNA using lipofectamine reagent (Invitrogen, Carlsbad, Calif., USA) according to the manufacturers protocol. Positive clones were selected using the neomycin phosphotransferase gene as a marker. Individual stable clones were expanded and maintained in geneticin (G-418) containing media. UMPK mRNA and protein levels were assayed in all of the clones using Q-RT-PCR and Western Blotting. 5-FU cytotoxicity was determined using the MTS assay (Promega, Madison, Wis., USA). At least five clones, each expressing UMPK cDNA or containing empty vector were assayed and three each were chosen for further analysis of 5-FU sensitivity using MTS assay and treatment protocols described below.

### UMPK Down-Regulation

Gene knockdown was performed using siRNA technology. Small interfering RNAs were designed for two different exons of the UMPK gene and synthesized by Ambion (Ambion, Austin, Tex., USA). HCT-8/P cells were transiently transfected with siRNA using oligofectamine reagent (Invitrogen, Carlsbad, Calif., USA) and a standard protocol. Briefly, cells were seeded at 0.25×10⁶ in cell culture medium without antibiotics the day before transfection in 6 well plates. Transfection was performed in OptiMEM serum free medium (Invitrogen, Carlsbad, Calif., USA) and fresh growth medium was added 12 hours after transfection. Final concentrations of siRNAs tested were 20 nM and 40 nM. Scrambled siRNA was used as a negative control (Ambion, Austin, Tex., USA). Cellular levels of UMPK mRNA and protein were analyzed by Q-RT-PCR and Western Blotting 24 h, 48 h and 72 h following the transfection in order to find the dose of siRNA and time post-transfection that resulted in effective knocking-down of UMPK. Subsequently, siRNA transfected cells were assayed for their sensitivity to bolus and continuous exposure to 5FU. Cells were subcultured 48 h after transfection.

### 5-FU/5-FUR Bolus Exposure

To mimic the clinical use of bolus 5-FU treatment in vitro conditions exponentially growing cells were exposed to 5-FU (0.1 μM-500 μM) or 5-FUR (0.001 μM-100 μM) for 4 hours (Sigma, St. Louis, Mo., USA). Then drug-containing medium was removed, cells were washed once with PBS and drug-free growth medium was added. Following the drug exposure, cells were cultured for 4 days before performing the MTS assay.

### 5-FU Continuous Exposure

To model the other clinically relevant use of 5-FU, namely, continuous infusions, exponentially growing cells were exposed to 5-FU (0.01 mM-100 μM) for 4 days. Number of viable cells was determined by MTS assay.

### Drug Treatment

5-Azadeoxycytidine (Sigma, St Louis, Mo., USA) was dissolved in DMSO and stored on ice until used. Exponentially growing cells were exposed to various low concentrations of the drug for 24 hours and than immediately treated with 5-FU or 5-FUR or harvested for DNA, RNA or protein analysis. 5-FU and 5-FUR (Sigma, St Louis, Mo., USA) solutions were prepared fresh before each use in culture media. Cells were exposed to 5-FU (0.1 μM-500 μM) or 5-FUR (0.001 μM-100 μM) for 4 hours. Then drug-containing medium was removed, cells were washed once with PBS and drug-free growth medium was added and were cultured for 4 days before performing the MTS assay.

### MTS Assay

Sensitivity of cells to 5-FU and 5-FUR was measured using the MTS assay (Promega, Madison, Wis., USA). Briefly, 2000 cells were plated in 96 well plates (Corning, One Riverfront Plaza, NY, USA) the day before addition of the drug. Dilutions of 5-FU and 5-FUR were prepared fresh in RPMI growth medium before each use. Manufacturer's protocol was followed for preparation and use of MTS reagent.

### Colorectal Cancer Patient Samples:

A total of 29 metastatic colorectal tumor samples from liver metastases were obtained from 29 different colorectal cancer patients. The tumors were obtained at the time of surgery, either for diagnostic or treatment purposes, with written informed consent. Diagnosis was made in the Department of Pathology, Memorial Hospital using a portion of the tumor specimen. Ultraspec reagent (Biotecx Labs, Houston, Tex.) was added to a viable portion of the tumor sample within minutes after removal, and flash frozen in liquid nitrogen till the time of analysis. Prior to analysis, tumors were ground to a powder in liquid nitrogen and both RNA and protein were extracted and Q-RT-PCR and Western Blotting were performed as described below with the following modifications. RNA was reverse transcribed using random primers (Invitrogen, Carlsbad, Calif., USA), and the product was used as template for subsequent PCR using Universal PCR Master Mix (Applied Biosystems, Foster City, Calif., USA) and LightCycler System (Roche, Indianapolis, Ind., USA). The relative UMPK mRNA level (y) was calculated according to the formula y=(100−x), where x=[(Ct_{UMPK}−Ct_{β-actin})/Ct_{β-actin}]^{*}100. The sequence of UMPK forward primer was 5′-TTG ACC CGT CTC CAT CGG-3′ (SEQ ID NO: 1), reverse primer 5′-TGC CTC CTG ACC CCT CCT-3′ (SEQ ID NO: 2) and probe 5′FAM-CCC CAG CCC CTA TCT CCA AGA GACA-3′TAMRA (SEQ ID NO: 3). The sequence of β-actin forward primers was 5′-TGA GCG CGG CTA CAG CTT-3′ (SEQ ID NO: 4), reverse primer 5′-TCC TTA ATG TCA CGC ACG ATT T-3′ (SEQ ID NO: 5) and probe 5′FAM-ACC ACC ACG GCC GAG CGG-3′TAMRA (SEQ ID NO: 6).

### Western Blotting

Cells (1-5×10⁶) were trypsinized, washed twice with PBS and kept frozen in −80° C. until used. Cell pellets or powdered tumors (vide infra) were thawed and resuspended in 0.5 ml of RIPA buffer (50 mM Tris-HCl buffer pH 7.4, 3 mM sodium fluoride and 4 mM DTT) supplemented with protease inhibitors. Samples were sonicated (2×20 s, 60% power) on ice and centrifuged for 20 min at 20,000 rpm. 50 mg of lysate was loaded onto 10% SDS-PAGE gel and after transfer to nitrocellulose probed with anti-UMPK polyclonal antibody (1:1000) for 2 hours (gift of Dr Y. C. Cheng, Yale University) and secondary ant-rabbit antibody (1:5000) for 1 hour (Sigma St Louis, Mo., USA) or anti-α-tubulin (1:10000) for 1 hour (Sigma, St Louis, Mo., USA) and anti-mouse antibody (1:2000) for 0.5 h (Santa Cruz, Calif., USA) as a control.

### Q-RT-PCR

Total RNA was extracted from 1-5×10⁶ cells or powdered tumors using Trizol Reagent (Invitrogen, Carlsbad, Calif., USA) and run on 1% folmaldehyde-agarose gel to assess the quality of the RNA. Gene specific primers and fluorescent-labeled probe (FAM and TAMRA) were designed using Primer Express Software from ABI (Applied Biosystems, Foster City, Calif., USA). 50 ng of RNA was amplified in one-step RT-PCR reaction (Applied Biosystems, Foster City, Calif., USA) using TaqMan Real-Time PCR machine (7000 SDS). 18SrRNA (FAM) (Applied Biosystems, Foster City, Calif., USA) or β-actin (FAM-TAMRA) was used to normalize the results. Delta delta Ct method was used to calculate relative fold change in mRNA level (Livak K J, Schmittgen T D. Analysis of relative gene expression data using real-time quantitative PCR and the 2(−Delta Delta C(T)) Method. Methods 2001 December; 25(4):402-8). The sequence of UMPK forward primer is 5′-AAG AAG GAA AGA TTG TAC CAG TTG AGA-3′ (SEQ ID NO: 7), reverse primer 5′-GGA AAC CCA TCA ATC AAG AAT TTA TT-3′ (SEQ ID NO: 8), and probe 5-FAM-AGA GGG AAA TGG ATC AGA CAA TGG CTG C-TAMRA-3′ (SEQ ID NO: 9).

### Cloning of the UMPK Promoter Region

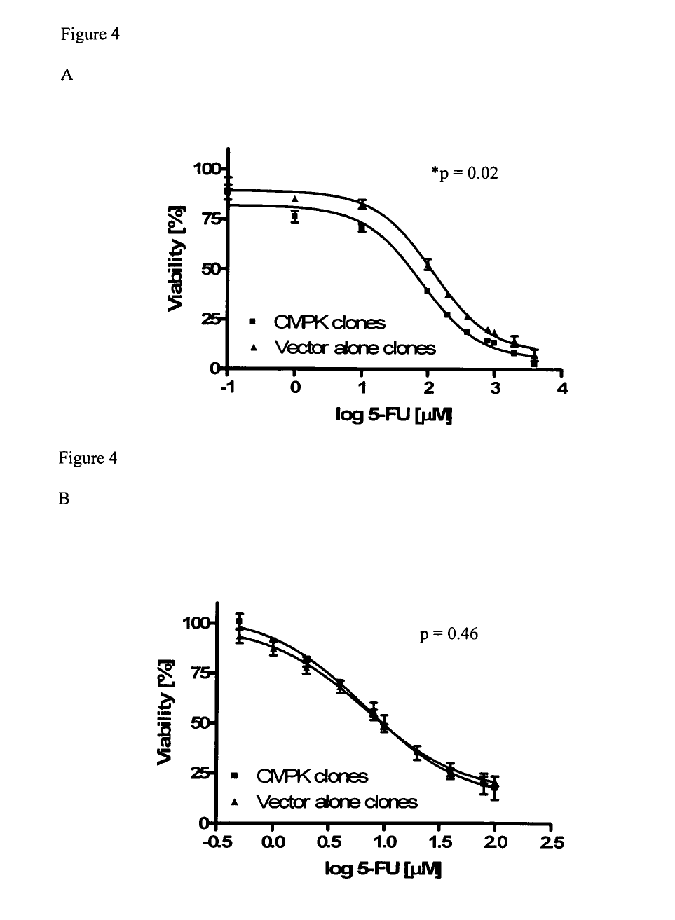

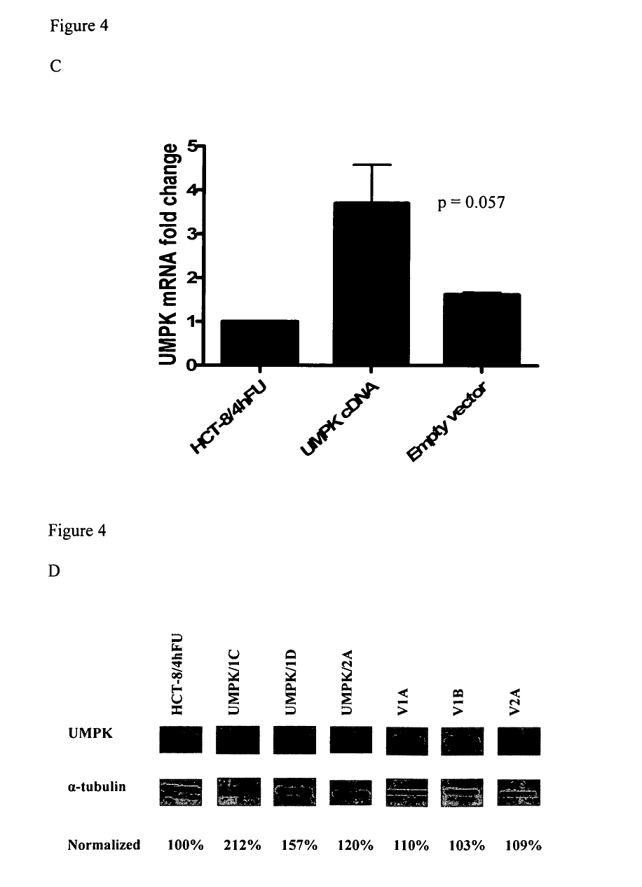

The sequence of 1000 bp and 2000 bp upstream of the UMPK translation initiation site was obtained from Ensemble Human Genome Data Resources at The Wellcome Trust Sanger Institute (FIG. 4A). Primers were designed to amplify these regions from HCT-8/P and HCT-8/4hFU cells: KpnIMluIUMPK/P2000FP-5′-TTTATTGGTACCACGCGTACAAAGGCAAGTAGGTGAGG-3′ (SEQ ID NO: 10), KpnIMluIUMPK/P1000FP-5′-GCGATTGGTACCACGCGTCTTTCCATA ATCAAAAGTCC-3′ (SEQ ID NO: 11), HindIIIXhoIUMPK/PRP 5′-AATA AAAAGCTTCTCGAGACACCGCGCCTCGGCCGGA-3′ (SEQ ID NO: 12). Products were cloned into pGL-3 basic and enhancer vectors (Promega, Madison, Wis., USA) that contain firefly luciferase gene as a reporter. Positive clones were confirmed by restriction digestion and sequencing. HCT-8/P and HCT-8/4hFU cells were transfected with the experimental constructs or an empty vector for background control (pGL3 basic, pGL3 enhancer). Cells were co-transfected with pRL-TK vector (renilla luciferase) (Promega, Madison, Wis., USA) in a 1:10 ratio as a control for transfection efficiency. pGL3 control vector containing SV-40 promoter was used as a positive control. Dual Luciferase Assay (Promega, Madison, Wis.) was performed 48 h after transfection and bioluminescence signals was recorded using luminometer TD-20/20 (Turner Design, DL Ready).

Determination of UMPK mRNA Decay

mRNA half life was determined using a method based on Real-Time RT-PCR analysis instead of a radioactive probe (Leclerc G J, Leclerc G M, Barredo J C. Real-time RT-PCR analysis of mRNA decay: half-life of Beta-actin mRNA in human leukemia CCRF-CEM and Nalm-6 cell lines. Cancer Cell Int 2002 Mar. 7; 2(1):1). Briefly, transcription was inhibited using Actinomycin D (5 μg/ml) or 5,6-dichlorobenzimidazole ribose (25 μM). Total RNA was extracted using Trizol Reagent (Invitrogen, Carlsbad, Calif., USA). RNA concentration was determined using RiboGreen fluorescent dye (Molecular Probes, Invitrogen, Carlsbad, Calif., USA). Quality and integrity of total RNA was assessed on 1% folmaldehyde-agarose gel. 50 ng of total RNA was amplified in one-step RT-PCR reaction (Applied Biosystems, Foster City, Calif., USA) using UMPK specific primers and probe (as described above). Serial dilutions of plasmid containing UMPK cDNA (gift of Dr. Y. C Cheng, Yale University) were used to prepare standard curve.

### Colorectal Cancer (CRC) Xenograft Model

HCT-8/P or HCT-8/4hFU cells (2.5×10⁶) were injected subcutaneously into SKID nu/nu mice (Taconic, Germantown, N.Y., USA). When tumors were palpable animals were randomized into 4 groups (5-10 animals per group) and treated with DAC (Tuesday, Wednesday, Thursday, 0.5 mg/kg, i.p.), or 5-FU (Friday, 50 mg/kg, i.p.), or DAC & 5-FU, or PBS as a control. Measurements of tumor size and animal weight were performed every 3-4 days. Two independent experiments were performed and animals received either 1 or 2 courses of treatment (1 or 2 weeks) followed by an additional week of observation. In order to assay UMPK induction in tumors, animals from DAC alone, and DAC & 5-FU groups received additional course of DAC treatment towards the end of experiment. 24 hours after the third dose of DAC was administered animals were anesthetized, and tumors harvested, washed in PBS and flash frozen in LN₂. Prior to the experiment tumors were grounded to a powder in LN₂. Following the tumor harvest or if tumor diameter is greater than 1 cm, or greater than 15% weight loss occurred the animals were euthanized. All experiments were performed according to the protocol approved by The Institutional Animal Care and Use Committee at RWJMS.

### Pyro Q-CpG Analysis of Methylation

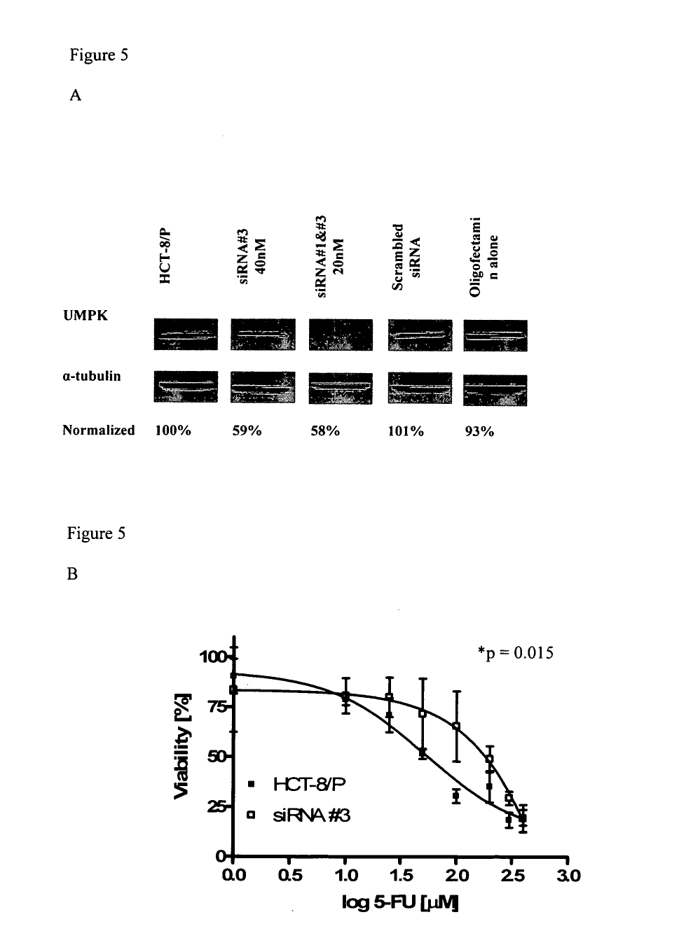

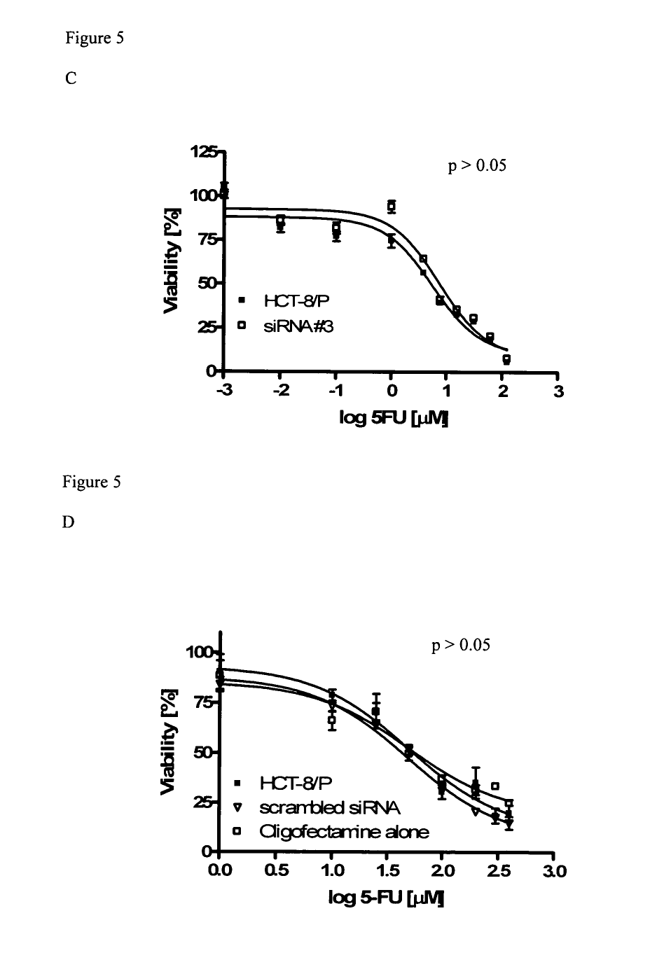

A search for CpG islands within the UMPK promoter was performed using web-based software EBI Tools/CpG Plot (Rice P, Longden I, Bleasby A. EMBOSS: the European Molecular Biology Open Software Suite. Trends Genet 2000 June; 16(6):276-7). By default, this program defines a CpG island as a region where the calculated percentage composition is over 50% and the calculated Observed/Expected ratio is over 0.6 and the conditions hold for a minimum of 200 bases. DNA was extracted from tumors or cells using standard phenol-chlorophorm-isoamyl alcohol extraction (Invitrogen, Carlsbad, Calif., USA). 2 μg of sample DNA or 1 μg of control DNA was bisulfite modified using EZ DNA Methylation Kit (Zymo Research, Orange, Calif., USA). 1 ul out of 10 ul of post bisulfite modified DNA was used for PCR. Region of UMPK promoter that was amplified is shown in FIG. 5B. Bisulfite treated fetal cell DNA and in vitro methylated human DNA (Chemicon, Millipore, Billerica, Mass.) were used in the assay development and PCR bias testing. The following reagents were mixed together in preparation for the Pyrosequencing reaction: Streptavidin beads 2 μl, 2× binding buffer 40 μl, PCR product 10 μl, water 28 μl. The assay was performed by EpigenDx (Worcester, Mass., USA) using PSQHS 96 System. Methylation of 42 CpG sites was analyzed. The average of r-square for all the CpG sites was 0.96 showing that this assay was biased slightly toward unmethylated DNA.

The following primers and cycling conditions were used:

(SEQ ID NO: 13)

FP-
5′-AATTATTTAAGGGTTGGAGGAAA-3′

(SEQ ID NO: 14)

RP-
5′biotin-CTACAACAAAAAACTAAAACCCAA-3′

(SEQ ID NO: 15)

Sequencing primer-
5′-TATTTAAGGGTTGGAGGAAAGT-3′
Cycling-
95° C. 15 min; 45 × (95° C. 30 s; 58° C. 30 s;
72° C. 30 s); 72° C. 10 min; 4° C.

### Statistical Analysis

GraphPad Prism 4 software (San Diego, Calif., USA) was used for statistical analysis and statistical significance was assayed using the student-T-test or repeated measure ANOVA in case of the analysis of delay in tumor progression. Gels were quantitated using ChemiDoc XRS machine and Quantity One 1-D analysis software (BioRad, Hercules, Calif., USA).

### Example 1

### Role of UMPK in the Resistance to 5-FU

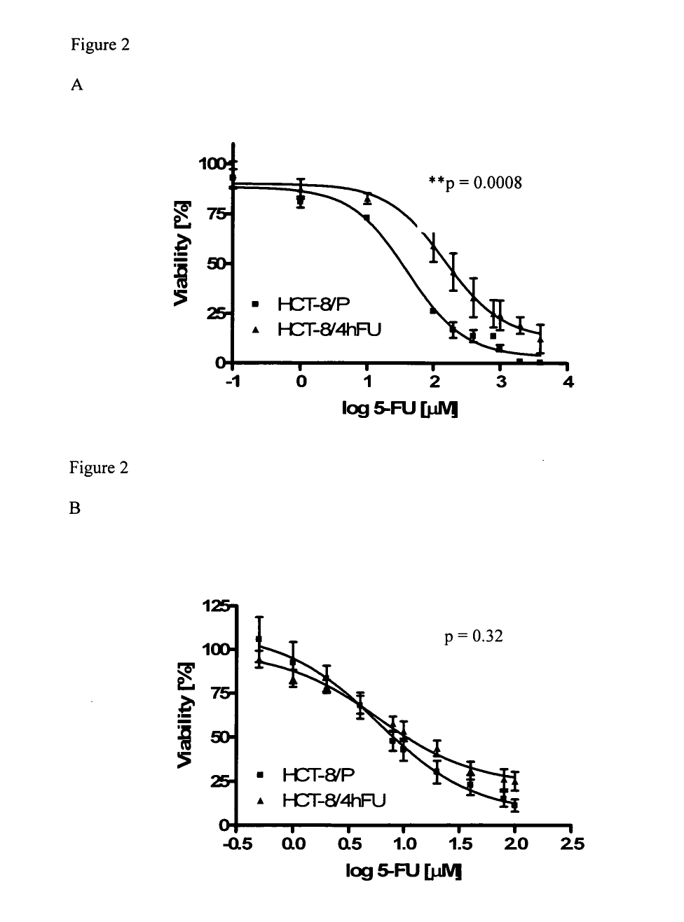

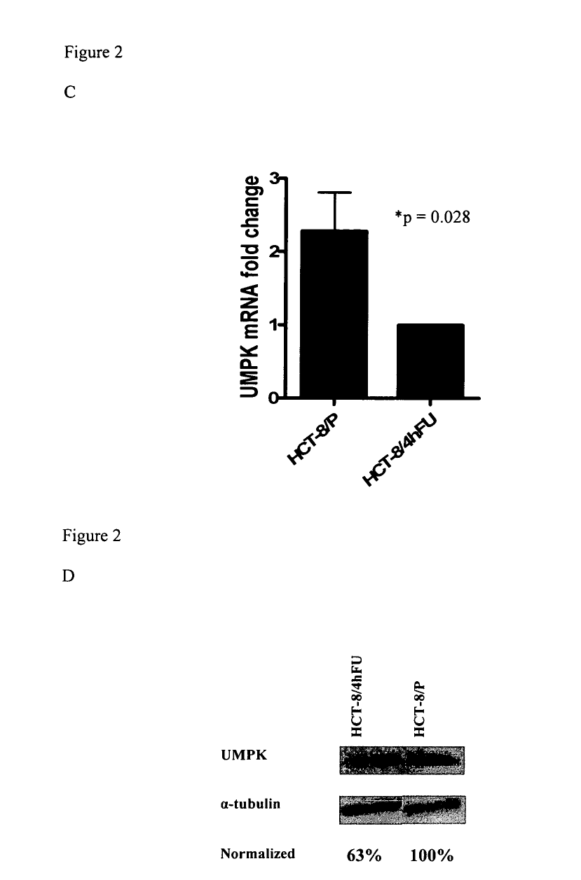

To study molecular mechanisms underlying resistance to pulse 5-FU, cells of the colorectal cancer cell line HCT-8 were exposed to repeated fixed doses of 5-FU that more closely mimics the situation under which the clinical resistance is developed. Resulting bolus 5FU resistant clones were denoted as HCT-8/4hFU and the parental cell line as HCT-8/P. Cell viability assays confirmed that HCT-8/4hFU cells are 3-4 fold more resistant to the treatment with bolus 5-FU but equally sensitive to continuous exposure to 5-FU (FIGS. 2A and 2B). Enzymatic activities of all major enzymes known to mediate 5-FU metabolism were assayed and activity of only UMPK was decreased (0.14±0.01 for HCT-8/P as compared to 0.09±0.02 for HCT-8/4hFU expressed in μmol/mg/min). There was no change in the Km for UMP between the resistant and the parental cell lines while the Vmax showed a 37% decrease (data not shown). Analysis of mRNA and protein levels of UMPK showed a decrease of 2-3 fold in resistant cells as compared to HCT 8/P (FIGS. 2C and 2D). While not intending to be bound by any theory or theories of operation, it is believed that these results indicate a reason for the previously reported lower incorporation of 5-FU into RNA in bolus resistant cells.

### Example 2

### Analysis of Colorectal Cancer Patient Samples

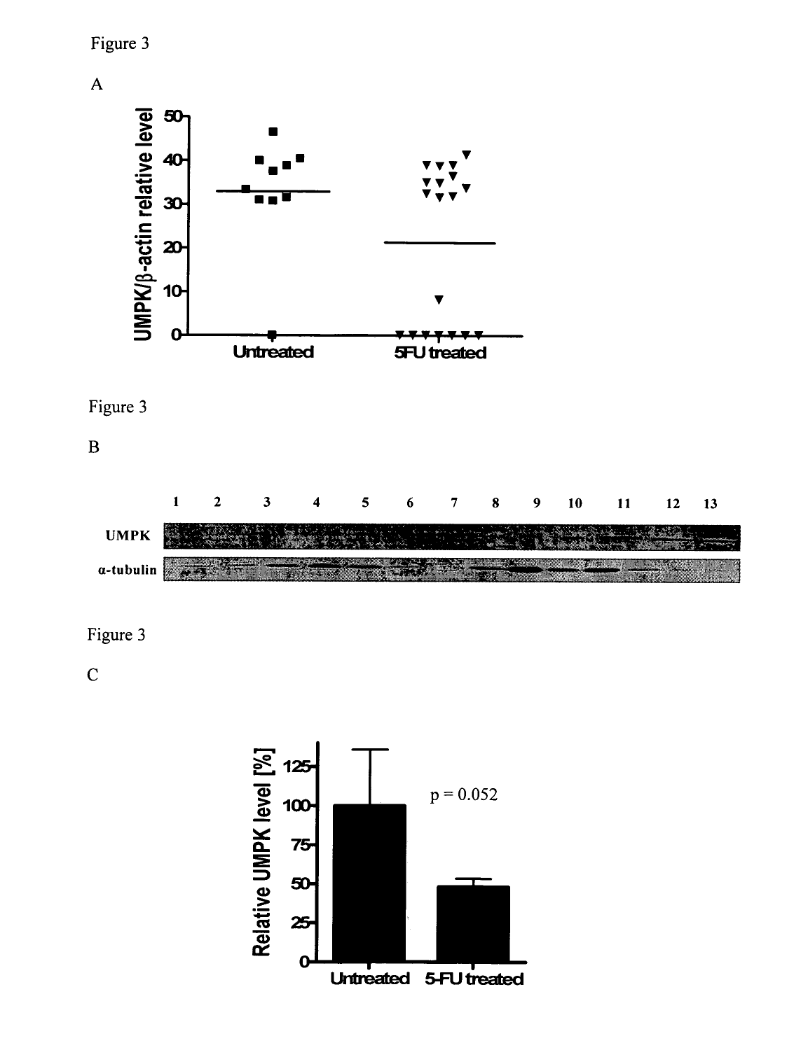

Twenty-nine (29) metastatic colorectal tumor samples were analyzed. Ten (10) were from patients that had not received any prior 5-FU treatment while the other 19 had received systemic 5-FU treatment weekly with leucovorin (Roswell Park Regimen). Seven of 19 previously treated patients had undetectable levels of UMPK mRNA and one sample had low levels of expression of UMPK. In the group of untreated patients, only one out of 10 tumors had no detectable UMPK mRNA (p=0.051) (FIG. 3A). Western blotting analysis on 13 of the 29 tumor samples for which lysates were available, confirmed that at the protein level UMPK expression in 5-FU treated samples were on average 2 fold lower as compared to the tumor samples from patients not previously treated with 5-FU (FIGS. 3B and 3C). While not intending to be bound by any theory or theories of operation, the data indicates that about 40% of patients that were previously exposed to 5-FU may have failed the treatment due to acquired resistance mediated in part by decreased expression of UMPK.

### Example 3

### Modulation of UMPK Levels by Up-Regulation

To further investigate the role of UMPK in resistance to bolus 5-FU, UMPK levels were modulated by overexpressing UMPK cDNA in bolus 5-FU resistant cells using mammalian expression vector pCR3.1. Stable clones were selected and assayed for their sensitivity to two clinically relevant 5-FU schedules, bolus and continuous treatments. UMPK cDNA transfected clones were more sensitive to bolus 5-FU as compared to empty vector transfected HCT-8/4hFU cells (FIG. 4A). They were equally sensitive to continuous 5-FU exposure (FIG. 4B). Levels of UMPK mRNA and protein in clones transfected with UMPK cDNA and empty vector were assayed and data is presented in FIGS. 4C and 4D. Although the pCR3.1 vector contains a strong CMV promoter that drives expression of a cloned gene, only moderate modulation in UMPK level was achieved. Taking into account that the experiment was repeated twice with similar results, while not intending to be bound by any theory or theories of operation, it is hypothesized that tight regulation of UMPK in the cell exists that prevents it from being highly up-regulated.

### Example 4

### Modulation of UMPK Levels by Down-Regulation

UMPK mRNA levels were down-regulated using siRNA in bolus 5-FU sensitive cells. Two different siRNAs were designed targeting exon 1 (siRNA#1) and exon 3 (siRNA#3) of the UMPK gene. Following the standardization of transfection efficiency, the 72 h time post-transfection was selected and siRNA#3 or a combination of siRNA#1 and siRNA#3 that resulted in a 50% decrease in UMPK protein (FIG. 5A) and mRNA levels (data not shown). Cytotoxicity assays revealed that down-regulation of UMPK confers resistance to bolus 5-FU treatment (FIG. 5B) but not to the continuous 5FU exposure (FIG. 5C). Moreover, scrambled siRNA or oligofectamine alone did not influence sensitivity of HCT-8/P cells to bolus 5-FU (FIG. 5D) or continuous 5-FU exposure (data not shown). These data further support the association of decreased levels of UMPK in acquired resistance to bolus 5-FU.

### Example 5

### Bolus 5-FU Resistant Cells Display Cross-Resistance to 5-Fluorouridine

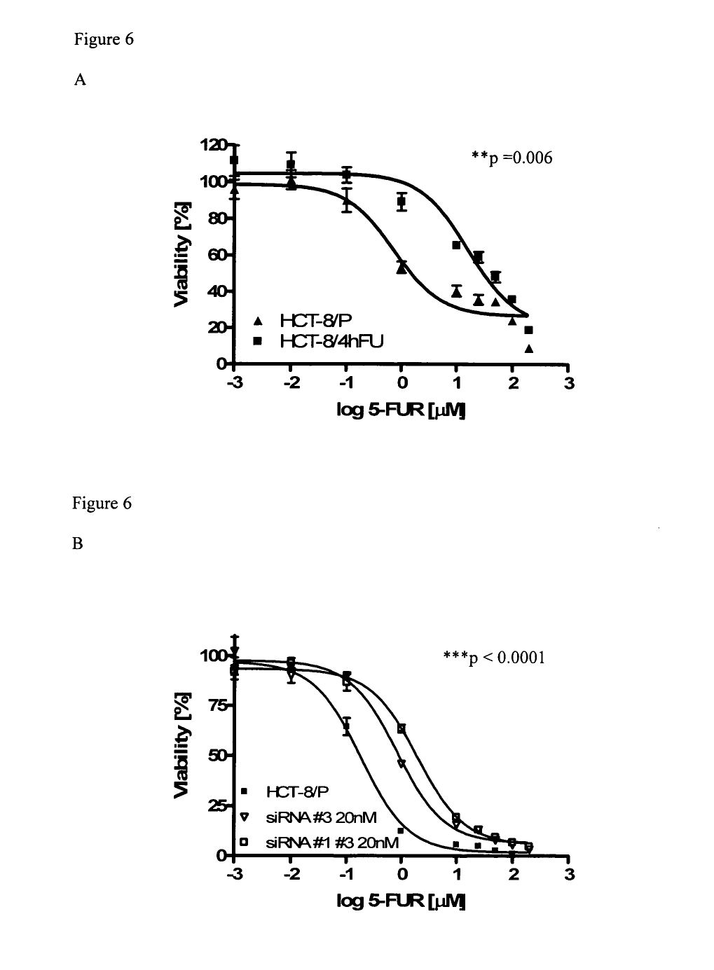

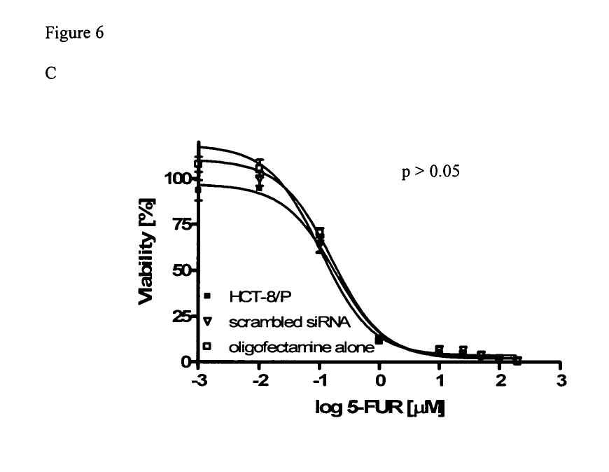

5-FUR is a 1β-D-ribofuranoside analog of 5-FU and its cytotoxic action is mainly RNA-directed (Wilkinson D S, Tlsty T D, Hanas R J. The inhibition of ribosomal RNA synthesis and maturation in Novikoff hepatoma cells by 5-fluorouridine. Cancer Res 1975 November; 35(11 Pt 1):3014-20). Similarly to bolus 5-FU, it requires the activity of UMPK to be activated to 5-FUTP that is incorporated into RNA. HCT-8/4hFU cells are also resistant to 5-FUR showing as high as 10-fold increase in the IC50 value as compared to HCT-8/P cells, as shown in FIG. 6A. Moreover, down-regulation of UMPK in HCT-8/P cells using siRNA #3 or combination of siRNA #1 and siRNA#3 induces resistance to 5-FUR (FIG. 6B). These results are consistent with the premise that bolus 5-FU administration is RNA directed and implicates UMPK in the development of the resistance to bolus 5-FU.

### Example 6

### Subcytotoxic Doses of DAC Modulates Response to Bolus 5-FU and 5-FUR In Vitro

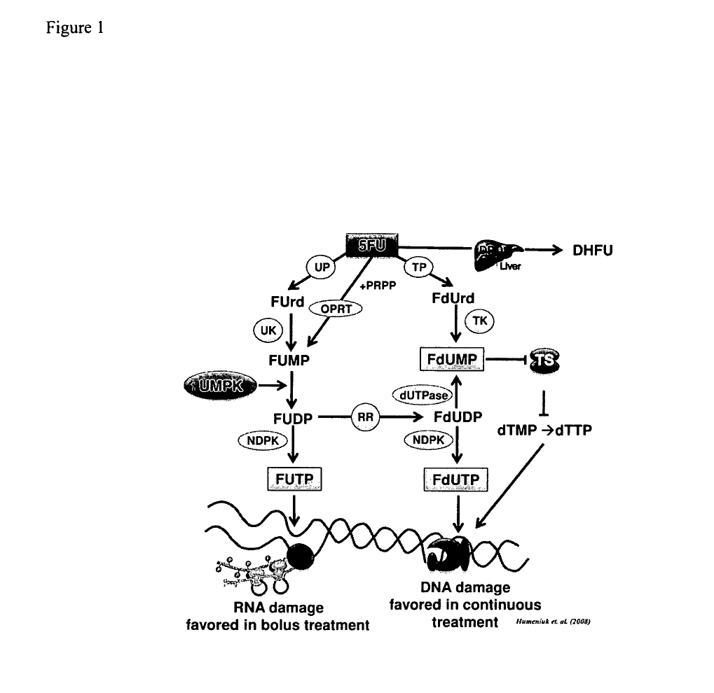

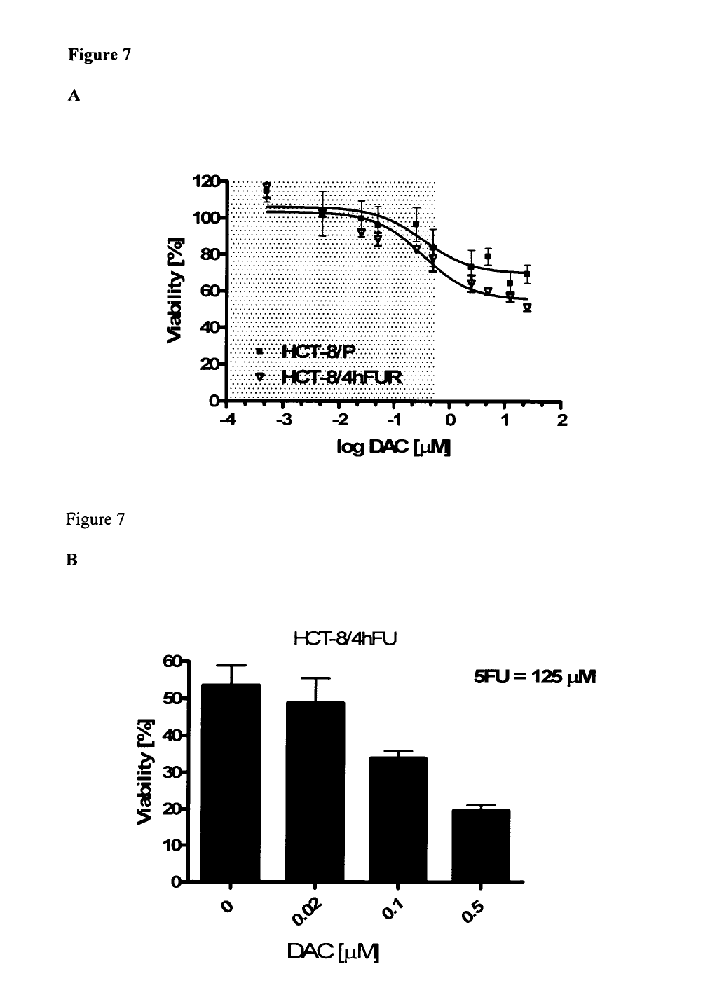

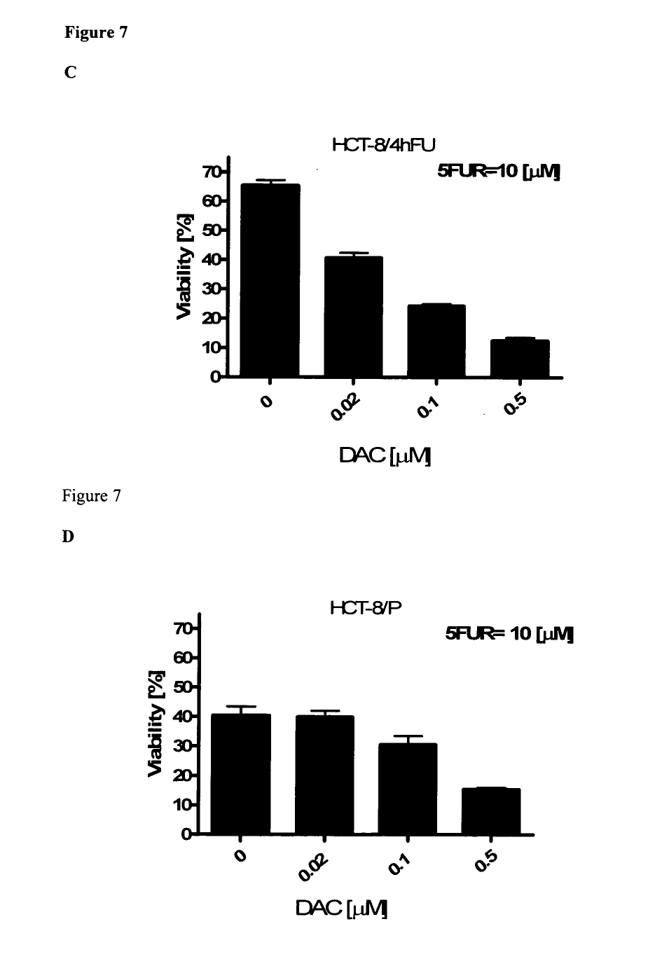

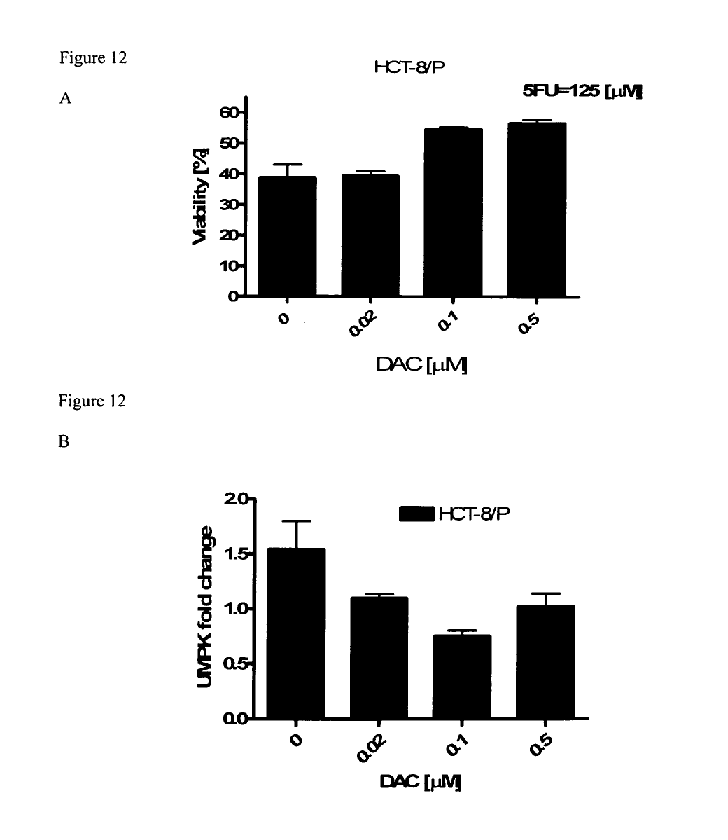

Low-doses of DAC are known to reactivate expression of genes silenced due to hypermethylation, while high doses are cytotoxic (Kantarjian H M, Issa J P. Decitabine dosing schedules. Semin Hematol 2005; 42: S17-22). Because of the dual mechanism of DAC action, its cytotoxicity profile towards HCT-8/P and HCT-8/4hFU cells was investigated (FIG. 1A). For further experiments doses between 0.0005 μM-0.5 μM were chosen that cause less than 20% of cell kill. The present inventors have found that HCT-8/4hFU cells are resistant to bolus 5-FU but sensitive to continuous 5-FU exposure and are cross-resistant to 5-FUR (Humeniuk R, Menon L G, Mishra P J, et al. Probing the role of Uridine Monophosphate Kinase in Fluoropyrimidine Resistance, Molecular Cancer Therapeutics 2008; submitted). 5-FUR and bolus 5-FU were previously shown to be predominantly incorporated into RNA and interfere with pre-rRNA processing. HCT-8/4hFU or HCT-8/P cells were pre-treated with 0.02, 0.01 or 0.05 μM DAC for 24 h and then exposed to high doses of 5-FU or 5-FUR for 4 hours. Cytotoxicity assays indicated that DAC pretreatment makes HCT-8/4hFU cells sensitive to bolus 5-FU treatment as well as 5-FUR treatment (FIGS. 7B and 7C). 0.02 μM DAC was able to restore the sensitivity of HCT-8/4hFU cells to that seen in HCT-8/P cells (FIG. 7D). Much less of an effect of DAC on sensitivity of HCT-8/P cells to 5-FU or 5-FUR was observed (FIG. 7D) and (FIG. 12A).

### Example 7

### DAC Increases Potency of Bolus 5-FU in Mouse Xenograft Model

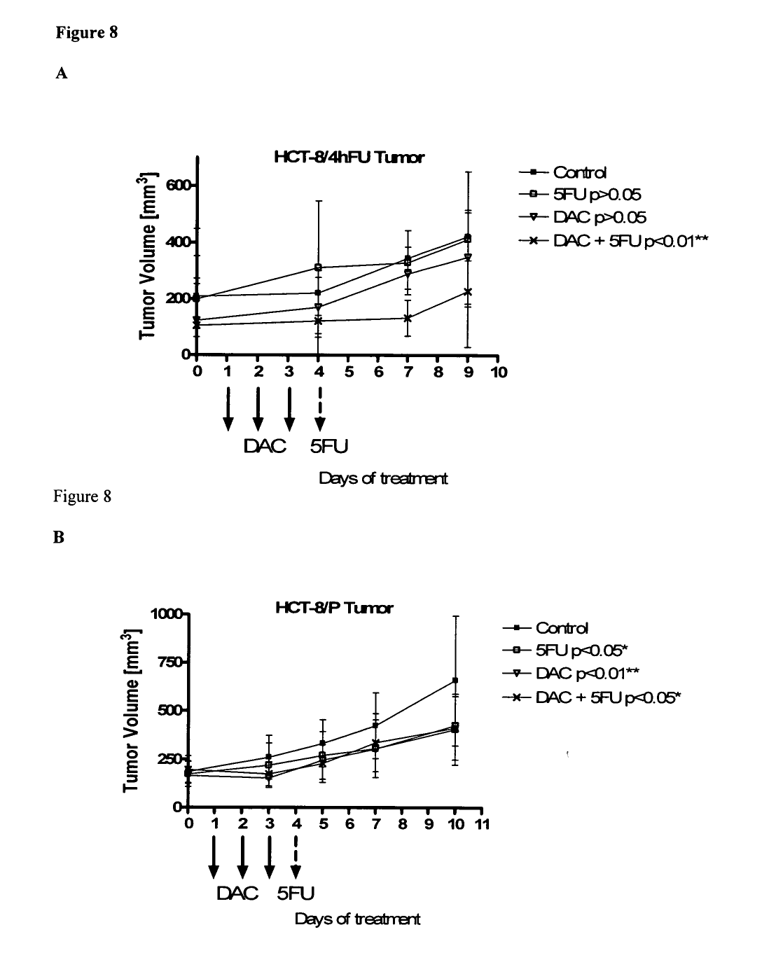

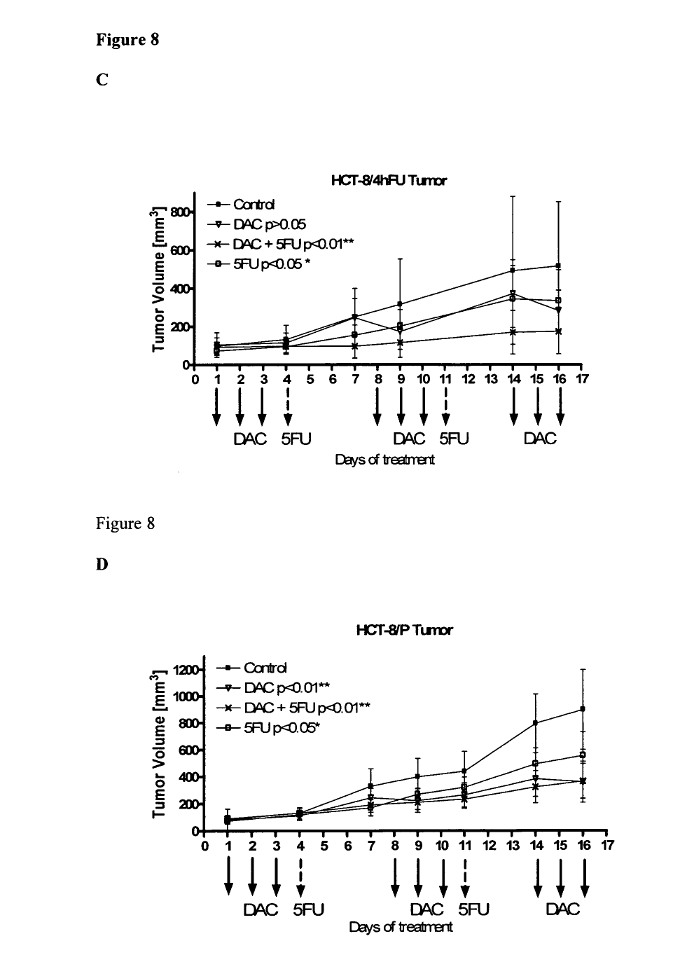

The effect of DAC on the response to bolus 5-FU was examined in a colorectal cancer xenograft model. HCT-8/4hFU and HCT-8/P cells were grown subcutaneously in nude mice. Animals were treated with low-dose DAC and bolus 5-FU as described in Materials and Methods. To assure reliability of the data two independent experiments were performed; mostly because of a high variability in tumor growth rates that occur the HCT-8/4hFU cell line. In the first experiment one course of treatment was administered as shown in FIGS. 8A and 8B. HCT-8/4hFU tumors were resistant to 5-FU alone or DAC alone treatments, but a significant reduction in the tumor volume was achieved when these drugs were given together (p<0.01). HCT-8/P tumors responded to all of the treatments administered with DAC alone treatment producing the most significant delay in the tumor growth (p<0.01). In the second experiment, two courses of treatment were administered followed by additional DAC treatment performed prior to tumor harvest and the results are shown in FIGS. 8C and 8D. Again, a significant delay in the HCT-8/4hFU tumor growth was observed in response to DAC treatment followed by bolus 5-FU treatment as compared to 5-FU alone (p<0.01). No significant response to treatment with DAC alone was observed and only a partial response to 5-FU alone was seen. HCT-8/P tumors, similar to the previous experiment, responded to all of the treatments administered, and DAC alone or when followed by 5-FU produced the most significant effect (p<0.01). Significant delay in the growth of the sensitive tumor produced by DAC alone in both of the experiments performed was unexpected. The utility of low-dose DAC in combination with 5-FU in the treatment of drug-resistant tumors as well as DAC alone in the treatment of drug-sensitive colorectal colorectal cancer tumors bears further study.

### Example 8

### Effect of DAC Treatment on UMPK Expression In Vitro and In Vivo

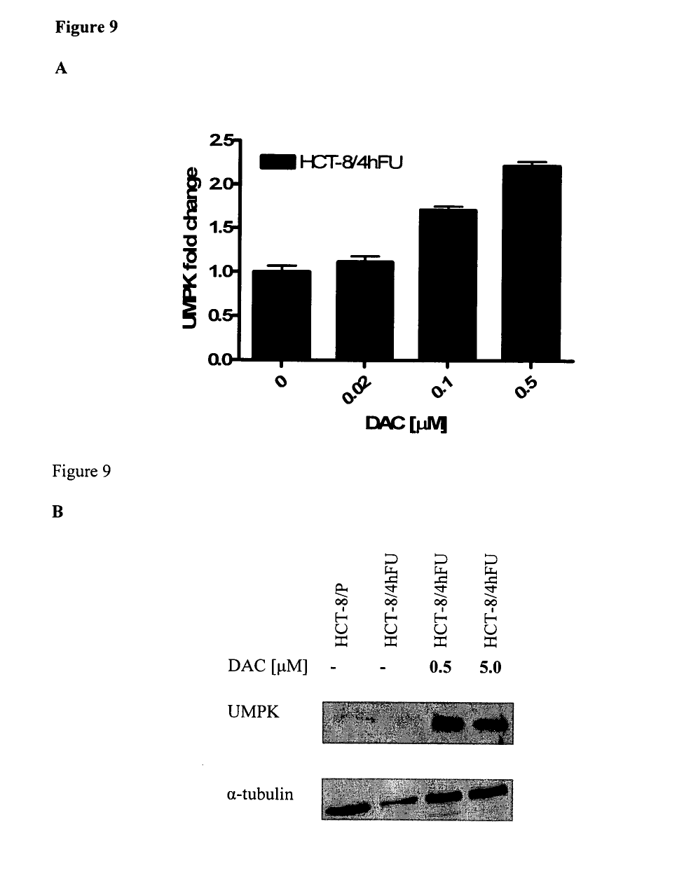

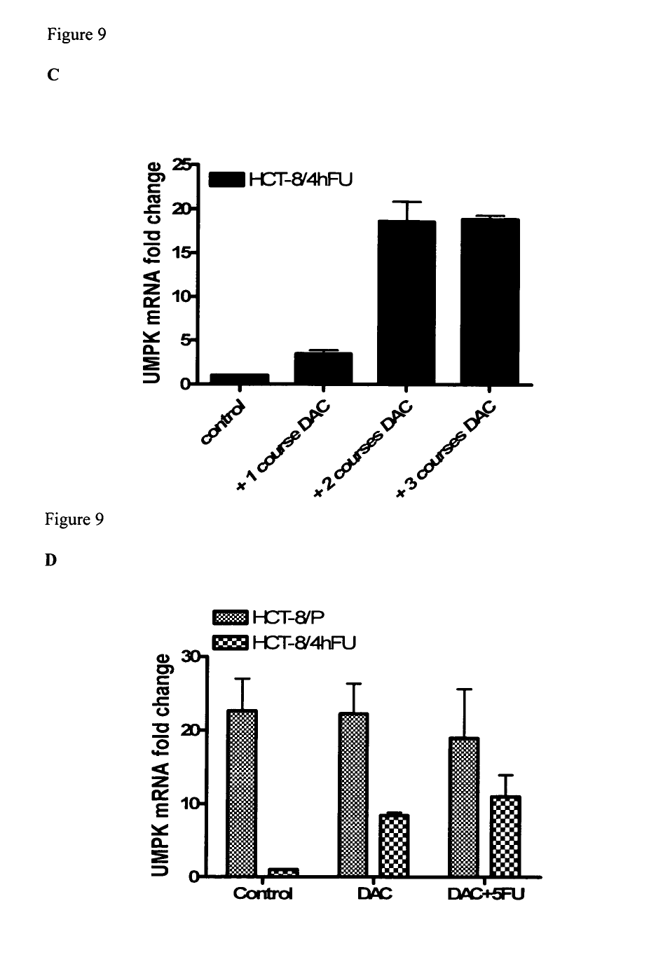

The present inventors have demonstrated herein that lower expression of UMPK confers resistance to bolus 5-FU and 5-FUR. The effect of DAC treatment on UMPK levels was investigated in the HCT-8/4hFU cells in vitro as well as in mouse xenografts bearing this tumor. A dose dependent increase in UMPK mRNA as well as protein levels was observed in HCT-8/4hFU cells treated with low-dose DAC for 24 h (FIGS. 9A and 9B). No increase in UMPK expression was observed in HCT-8/P cells (FIG. 9B). Analysis of mRNA isolated from tumors harvested at different time points following DAC administration showed a 4-fold increase in UMPK mRNA after one course of DAC treatment and up to a 20 fold increase after two and three weeks of treatment as compared to untreated tumor (FIG. 9C). The average increase of UMPK in the group of animals in tumors was 8.5 fold in the DAC alone group and 11 fold in the DAC+5FU group (FIG. 9D). No increase of UMPK after DAC treatment was observed in the HCT-8/P group. HCT-8/P tumors had on average, 20 fold more UMPK mRNA than HCT-8/4hFU as compared to only a 2-fold difference observed in the cultured cells.

### Example 9

### Cloning of UMPK Promoter Region

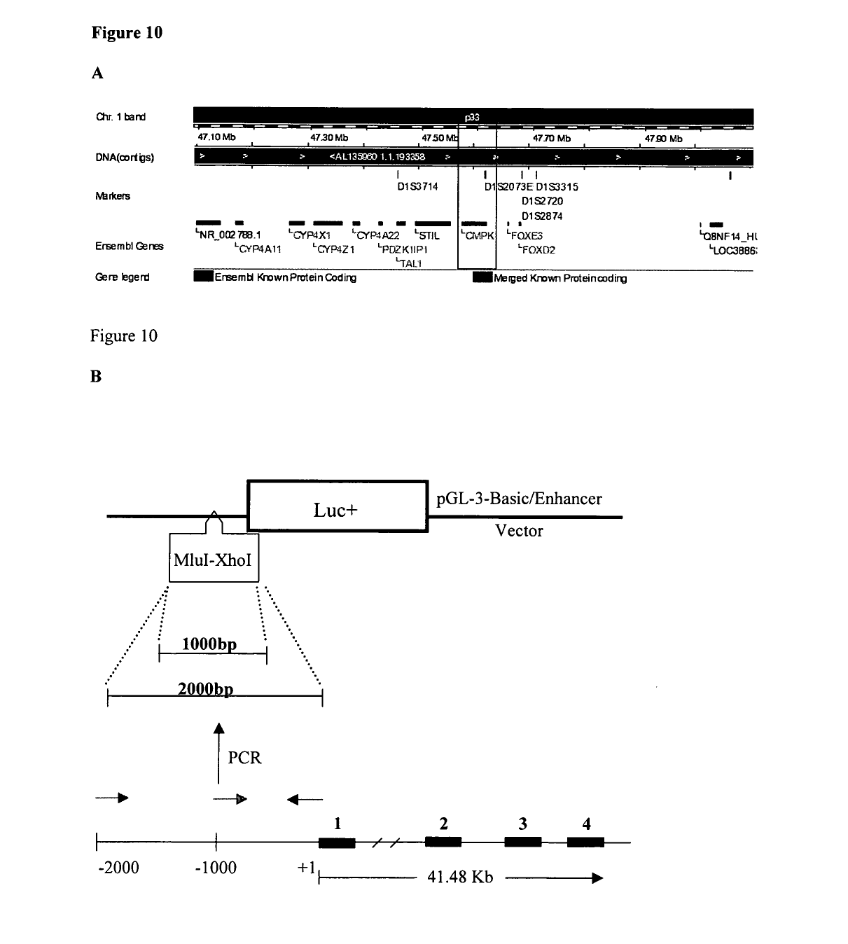

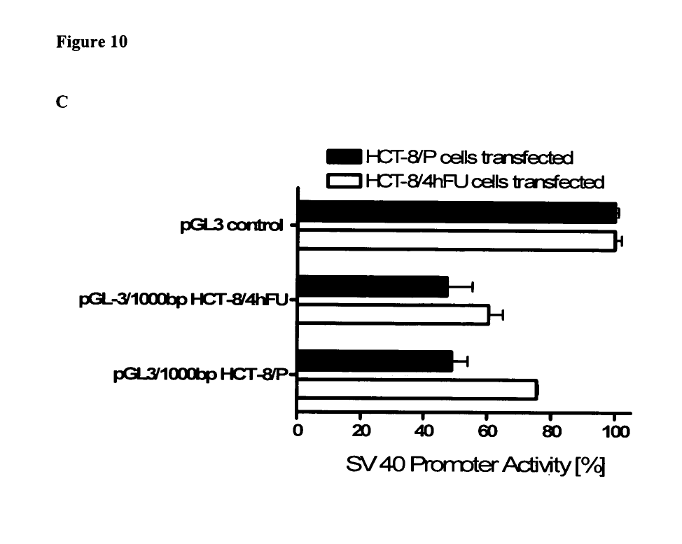

To study the mechanism of UMPK down-regulation in the HCT-8/4hFU cells and its reactivation mediated by DAC treatment, the putative UMPK promoter was cloned. 1000 bp and 2000 bp regions upstream of known UMPK translation start site was amplified from genomic DNA extracted from both HCT-8/P and HCT-8/4hFU cells and cloned into pGL-3 vectors as shown in FIGS. 10A and 10B. Sequencing of the cloned regions did not reveal any significant changes besides 3 SNPs of unknown function (data not shown). Expression of pGL-3 constructs in HCT-8/P cells showed that activities of the putative promoters are about 50% of the SV40 promoter activity and are the same for regions amplified from HCT-8/P as well as HCT-8/4hFU cell lines (FIG. 10C). Expression of promoter constructs in the HCT-8/4hFU cells showed an increased activity for both constructs. The activity of the construct-containing region amplified from HCT-8/P cells, had slightly higher activity than that from HCT-8/4hFU. No significant difference between constructs containing 1000 bp or 2000 bp cloned into pGL-3 basic or enhancer vectors was found (data not shown). This data suggests that no functional changes between promoter regions exist between the sensitive and resistant cell lines.

### Example 10

### Analysis of UMPK Promoter Methylation

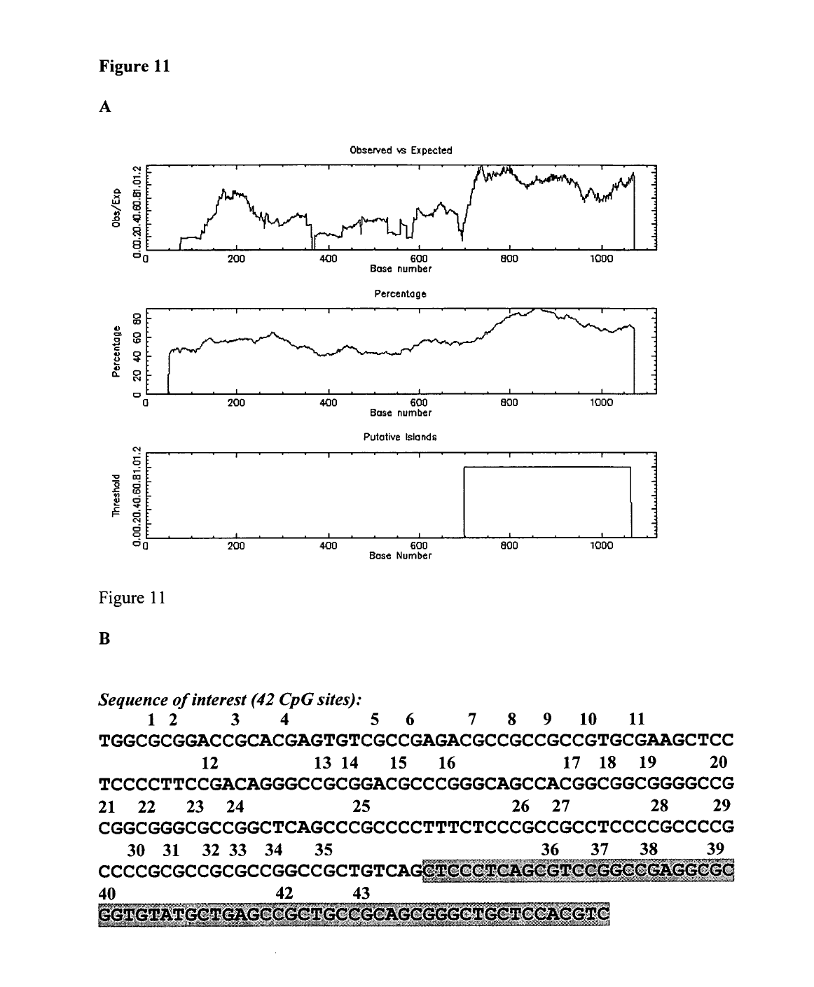

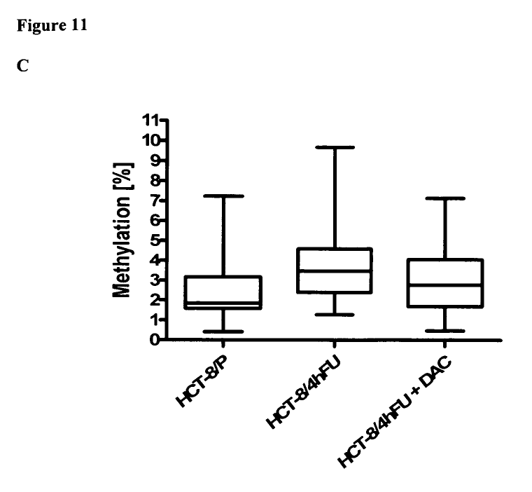

Scanning of cloned 1000 bp of UMPK promoter region identified one CpG island surrounding the putative transcription start site (FIG. 11A). A 240 bp region (SEQ ID NO: 16) (sequence and genomic context is shown in FIG. 11B) was chosen for further analysis of DNA methylation. A total of 42 CpG sites were analyzed within this sequence and 36 gave reliable results (verified by mixing studies). Enrichment in UMPK promoter DNA methylation was observed in HCT-8/4hFU cells as compared to HCT-8/P cells. Moreover, treatment of HCT-8/4hFU cells with DAC resulted in the reversal of DNA methylation. An average and a median DNA methylation plus/minus standard deviation across 36 sites for HCT-8/P, HCT-8/4hFU and HCT-8/4hFU cells treated with 0.5 μM DAC for 24 h is shown in FIG. 5C. Mean methylation is 1.85, 3.47 and 2.74 for HCT-8/P, HCT-8/4hFU and HCT-8/4hFU +DAC, respectively. Methylation at individual sites for both of the cell lines before and after treatment with DAC is shown in Table 1 (below). Analysis using the paired t-test showed that observed changes in DNA methylation are statistically significant (p<0.0001). Thus, a decrease in the UMPK mRNA level in the HCT-8/4hFU cells may be due to an increase in DNA methylation of the UMPK promoter that interferes with binding of transcription factors.

TABLE 1
Methylation of individual CpG sites analyzed within UMPK
promoter and first exon

| | | | | | HCT-8/ | | HCT-8/4hFU + | |
|---|---|---|---|---|---|---|---|---|
| | HCT-8/P | | P + DAC | | 4hFU | | DAC | |
| CpG | Mean | SD | Mean | SD | Mean | SD | Mean | SD |
| 1 | 1.59 | 0.10 | 1.53 | 0.12 | 1.42 | 0.21 | 1.28 | 0.11 |
| 2 | 1.34 | 0.33 | 2.02 | 0.01 | 1.28 | 0.12 | 0.46 | 0.46 |
| 3 | 1.81 | 0.09 | 2.52 | 0.22 | 1.89 | 0.11 | 1.67 | 0.00 |
| 4 | 0.42 | 0.42 | 2.36 | 0.71 | 1.90 | 0.15 | 2.36 | 0.17 |
| 5 | 1.74 | 0.18 | 2.60 | 0.08 | 2.71 | 0.11 | 1.53 | 0.35 |
| 6 | 1.44 | 0.18 | 0.59 | 0.59 | 2.81 | 0.10 | 1.65 | 0.20 |
| 7 | 0.48 | 0.48 | 1.52 | 0.38 | 1.69 | 0.07 | 1.09 | 1.09 |
| 8 | 2.18 | 0.11 | 1.75 | 0.27 | 2.76 | 0.19 | 1.74 | 0.56 |
| 9 | 2.27 | 0.10 | 2.10 | 0.59 | 3.44 | 0.49 | 1.54 | 0.27 |
| 10 | 1.82 | 0.19 | 1.93 | 0.17 | 3.97 | 0.11 | 1.95 | 0.12 |
| 11 | 0.65 | 0.65 | 1.83 | 0.47 | 1.92 | 0.17 | 1.70 | 0.58 |
| 17 | 2.96 | 0.10 | 3.22 | 0.08 | 4.07 | 0.33 | 4.06 | 0.20 |
| 18 | 1.75 | 1.75 | 1.52 | 1.52 | 1.70 | 1.70 | 3.07 | 1.04 |
| 19 | 3.50 | 1.42 | 2.23 | 2.23 | 4.00 | 1.72 | 3.01 | 3.01 |
| 20 | 2.57 | 2.57 | 3.98 | 0.56 | 5.78 | 0.19 | 4.53 | 1.14 |
| 21 | 1.54 | 1.54 | 1.70 | 1.70 | 2.25 | 2.25 | 1.74 | 1.74 |
| 22 | 1.69 | 1.69 | 2.96 | 0.98 | 3.96 | 0.77 | 3.10 | 1.14 |
| 23 | 1.52 | 1.52 | 2.66 | 0.50 | 2.96 | 0.63 | 1.48 | 1.48 |
| 24 | 1.76 | 1.76 | 3.49 | 0.69 | 3.90 | 0.38 | 3.16 | 0.32 |
| 25 | 4.46 | 0.81 | 4.90 | 0.73 | 5.87 | 0.60 | 5.59 | 1.54 |
| 26 | 6.66 | 0.27 | 6.76 | 0.73 | 7.87 | 0.40 | 6.81 | 0.86 |
| 27 | 1.02 | 1.02 | 3.19 | 0.28 | 3.01 | 0.16 | 1.64 | 1.64 |
| 28 | 2.28 | 2.28 | 4.49 | 0.87 | 5.40 | 0.32 | 5.27 | 0.17 |
| 29 | 1.58 | 1.58 | 3.84 | 0.22 | 4.20 | 0.49 | 1.78 | 1.78 |
| 30 | 3.88 | 0.00 | 5.01 | 0.00 | 4.24 | 0.00 | 4.79 | 0.00 |
| 32 | 2.90 | 0.00 | 3.50 | 0.00 | 4.73 | 0.00 | 3.27 | 0.00 |
| 33 | 1.84 | 0.00 | 2.67 | 0.00 | 2.70 | 0.00 | 2.02 | 0.00 |
| 34 | 2.41 | 0.00 | 3.04 | 0.00 | 3.45 | 0.00 | 3.75 | 0.00 |
| 35 | 2.69 | 0.00 | 3.33 | 0.00 | 3.48 | 0.00 | 2.81 | 0.00 |
| 36 | 7.24 | 0.00 | 7.80 | 0.00 | 9.68 | 0.00 | 7.14 | 0.00 |
| 37 | 3.67 | 0.00 | 5.19 | 0.00 | 5.71 | 0.00 | 4.03 | 0.00 |
| 38 | 1.58 | 0.00 | 1.73 | 0.00 | 2.47 | 0.00 | 2.72 | 0.00 |
| 39 | 3.38 | 0.00 | 4.25 | 0.00 | 4.44 | 0.00 | 3.68 | 0.00 |
| 40 | 1.86 | 0.00 | 2.26 | 0.00 | 2.33 | 0.00 | 2.20 | 0.00 |
| 41 | 5.68 | 0.00 | 6.31 | 0.00 | 7.84 | 0.00 | 6.68 | 0.00 |
| 42 | 4.55 | 0.00 | 4.56 | 0.00 | 5.21 | 0.00 | 4.57 | 0.00 |

### Example 11

### UMPK mRNA Half-Life Studies

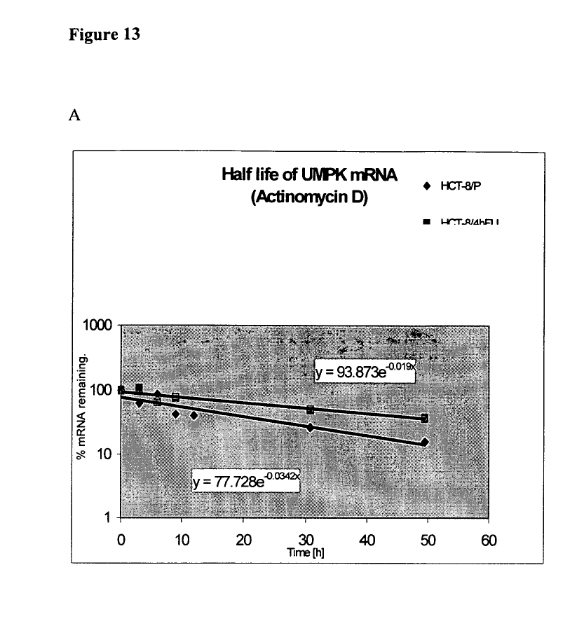

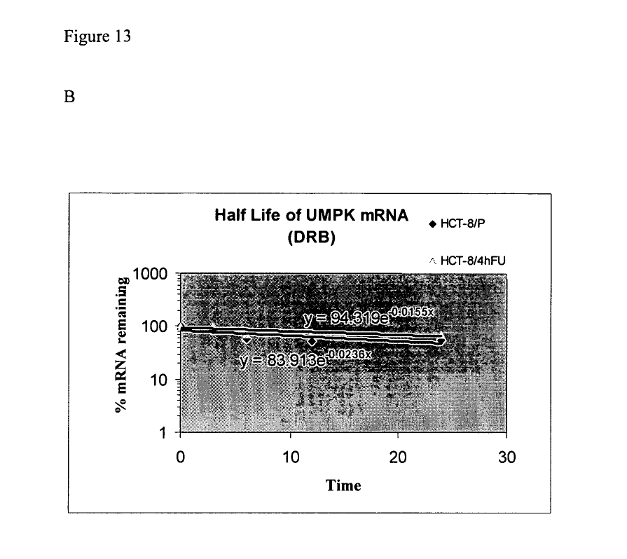

To exclude the possibility that observed decrease in UMPK mRNA and protein levels is HCT-8/4hFU cells is due to decreased message stability, half life studies were carried out. Two different transcription inhibitors were used and both showed that relative UMPK half-life is even longer in HCT-8/4hFU cells (34 h) than in HCT-8/P cells (20 h) (FIGS. 13A and 13B).

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

The terms and expressions which have been employed are used as terms of descriptions and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope on this invention.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

The disclosures of each patent, patent application and publication cited or described in this document are hereby incorporated herein by reference, in their entirety.

What is claimed is:

1. A method for the treatment of cancer which comprises administering to a subject in need thereof a therapeutically effective amount of a) a DNA methylation inhibitor and b) an antineoplastic agent.

2. The method of claim 1 wherein the DNA methylation inhibitor is selected from the group consisting of 5-azacytidine, 5-azadeoxycytidine, zebularine, epigallocatechin-3-gallate, 4-aminobenzoic acid derivatives, psammaplins and mixtures thereof.

3. The method of claim 1 wherein the DNA methylation inhibitor is 5-azadeoxycytidine.

4. The method of claim 1 wherein the antineoplastic agent is a fluoropyrimidine.

5. The method of claim 4 wherein the fluoropyrimidine is selected from the group consisting of 5-fluorouracil, 5-fluorouridine, and mixtures thereof.

6. The method of claim 4 wherein the fluoropyrimidine is administered on a bolus dosing schedule.

7. The method of claim 1 wherein the cancer is selected from the group consisting of colorectal cancer, gastric cancer, and breast cancer.

8. A method for increasing the efficacy of a nucleoside analog comprising the step of administering to a subject in need thereof a therapeutically effective amount of a) a DNA methylation inhibitor and b) the nucleoside analog.

9. The method of claim 8 wherein the nucleoside analog is selected from the group consisting of 5-fluorouracil, 5-fluorouridine, zebularine, 1-β-D-arabinofuranosylcytosine (AraC), 2′,2′-difluorodeoxycytidine (gemcitibine), β-D-2′,3′-dideoxycytidine (ddC), β-L-2′,3′-dideoxy-3′-thiocytidine (3-TC), 2′3′-deoxy-3′-azidothymidine (AZT), and mixtures thereof.

10. A method to develop a prognosis for or diagnose a subject's development of resistance to treatment with an antineoplastic agent, the method comprising the steps of:
a) obtaining a sample from the subject;
b) measuring in the sample the level of expression of UMPK; and
c) comparing the level of expression of UMPK of the sample with that of a standard.

11. The method of claim 10, wherein the standard level of expression of UMPK is selected from the group consisting of: the level of expression of UMPK in a sample obtained from a healthy part of the subject's body, a sample obtained from a healthy individual, a previous sample obtained from the subject, and known levels of UMPK expression in a healthy individual.

12. The method of claim 10 wherein the level of expression of UMPK in the sample is measured by assaying the amount of UMPK RNA in the sample.

13. The method of claim 10 wherein the level of expression of UMPK in the sample is measured by assaying the amount of UMPK protein in the sample.

14. A method of diagnosing the development of resistance to treatment with a nucleoside analog in a subject comprising the steps of:
a) obtaining a first sample from the subject;
b) measuring in the sample the level of expression of UMPK;
c) administering at least one nucleoside analog to the subject;
d) obtaining a second sample from the subject;
e) measuring in the second sample the level of expression of UMPK; and
f) comparing the level of expression of UMPK of the first sample with that of the second sample.

15. A composition for use in treating cancer comprising:
a) a DNA methylation inhibitor and b) an antineoplastic agent.

16. The composition of claim 15 wherein the DNA methylation inhibitor is selected from the group consisting of 5-azacytidine, 5-azadeoxycytidine, zebularine, epigallocatechin-3-gallate, 4-aminobenzoic acid derivatives, psammaplins and mixtures thereof.

17. The composition of claim 15 wherein the antineoplastic agent is a fluoropyrimidine.

18. The composition of claim 15 further comprising a liposome.

19. A method for making a composition useful for treating cancer comprising the step of combining a) a DNA methylation inhibitor and b) an antineoplastic agent.

20. The method of claim 19, further comprising the step of encapsulating the DNA methylation inhibitor and antineoplastic agent in liposomes.
